(19) United States
(12) Patent Application Publication
(10) Pub. No.: US 2020/0270664 A1
(43) Pub. Date: Aug. 27, 2020

(54) STABILIZED REDUCING AGENTS AND METHODS USING SAME

(71) Applicant: 10X Genomics, Inc., Pleasanton, CA (US)

(72) Inventors: Lawrence Greenfield, San Mateo, CA (US); Christopher Hindson, Pleasanton, CA (US)

(21) Appl. No.: 16/809,280

(22) Filed: Mar. 4, 2020

Related U.S. Application Data
(63) Continuation of application No. 15/156,628, filed on May 17, 2016.
(60) Provisional application No. 62/163,298, filed on May 18, 2015.

(51) Int. Cl. C12P 19/34; C07C 323/41; C12Q 1/6806; C12Q 1/6848

(52) U.S. Cl. CPC C12P 19/34; C12Q 1/6848; C12Q 1/6806; C07C 323/41

(57) ABSTRACT

The disclosure provides stabilized reducing agents and methods for using them in sample preparation. Stabilized reducing agents described herein provide easy-to-use replacement reducing agents for reducing agents that undergo side-reactions that can render them ineffective as reducing agents and/or decrease the concentration of available reducing agent. In some cases, a stabilized reducing agent is an activatable reducing agent that can be activated upon application of a stimulus to the reducing agent.

### CROSS-REFERENCE

This application is a continuation of U.S. application Ser. No. 15/156,628, filed May 17, 2016, which claims priority to U.S. Provisional Patent Application No. 62/163,298, filed May 18, 2015, which applications are herein incorporated by reference in their entirety for all purposes.

### BACKGROUND

Many critical reactions are highly sensitive to the concentrations of the various reagents present within the reaction mixture such that an effective reaction may only be achieved when certain reactants are present within a very narrow concentration window. In some cases, this may be due to stoichiometric chemistries involved in the reactions, where concentrations of a particular reagent beyond that required to react may be toxic. In other cases, the transient nature of a given reagent, e.g., instability, volatility, or cross-reactivity, may require its presence in a reaction at a given concentration at a particular moment in time.

Difficulties can arise in carrying out chemical processes using these transient reagents, as they can require preparation of fresh reagents before each use, require use in highly controlled, and potentially sub-optimal conditions, and/or result in extreme difficulties in transferring reaction processes to others, e.g., customers, through reaction kits and the like.

### SUMMARY

It can be desirable to be able to provide replacement reagents for transient reagents by providing stabilized reagent compositions that can more accurately provide a desired concentration of a given reactant, while concurrently providing it in a robust and forgiving form that is easily used by researchers of widely disparate levels of sophistication. The present disclosure addresses these and other needs.

The present disclosure provides stabilized reducing agents and methods for using them. The stabilized reducing agents can be useful in a variety of contexts, including sample preparation. Moreover, stabilization of a reducing agent can render it an activatable reducing agent. Such a stabilized, activatable reducing agent can be activated upon application of a stimulus to the reducing agent.

An aspect of the disclosure provides a method for nucleic acid amplification. The method comprises providing a reagent in a reaction volume, where the reagent comprises a first component coupled to a second component through a reducible linkage, and where the reaction volume includes a template nucleic acid molecule and a stabilized reducing agent; cleaving the reducible linkage with the aid of the stabilized reducing agent to decouple the first component from the second component; and subjecting the template nucleic acid molecule to an amplification reaction, using the first component, to yield nucleic acid molecules as amplification products of the template nucleic acid molecule. In some cases, prior to subjecting the template nucleic acid molecule to an amplification react, the reducing activity of the stabilized reducing agent with respect to the reducible linkage may vary by at most 20%. In some cases, prior to subjecting the template nucleic acid molecule to an amplification react, the reducing activity of the stabilized reducing agent with respect to the reducible linkage may vary by at most 10%. In some cases, prior to subjecting the template nucleic acid molecule to an amplification react, the reducing activity of the stabilized reducing agent with respect to the reducible linkage may vary by at most 5%. In some cases, prior to subjecting the template nucleic acid molecule to an amplification react, the reducing activity of the stabilized reducing agent with respect to the reducible linkage may vary by at most 1%. Moreover, in some cases, subjecting the template nucleic acid molecule to the amplification reaction may include cycling the temperature of the reaction volume. In some cases, after subjecting the template nucleic acid molecule to the amplification, the method further comprises providing a sequence of at least a portion of the amplification products.

In some cases, the stabilized reducing agent comprises a stabilized thiol containing compound. In some cases, the stabilized thiol containing compound comprises a sterically hindered thiol group. In some cases, the stabilized thiol containing compound comprises penicillamine. In some cases, the second component comprises a bead.

In some cases, the first component comprises a nucleic acid molecule and the second component comprises a macromolecular matrix. In some cases, the macromolecular matrix comprises a polymer matrix. In some cases, the polymer matrix comprises a hydrogel. In some cases, the hydrogel comprises cross-linked polyacrylamide. In some cases, the cross-linked polyacrylamide is cross-linked by a reducible cross-linkage and the method can further comprise cleaving the reducible cross-linkage prior to subjecting the template nucleic acid molecule to the amplification reaction. In some cases, the reducible cross-linkage comprises a disulfide linkage.

In some cases, the reducible linkage comprises a disulfide linkage linking the nucleic acid molecule to the polymer matrix. In some cases, the nucleic acid molecule comprises a barcode sequence. In some cases, the amplification products comprise the barcode sequence. In some cases, the amplification products comprise a partial hairpin structure.

In some cases, the reaction volume is provided in the aqueous interior of a droplet in a water-in-oil emulsion. In some cases, the reaction volume comprises an enzyme, such as, for example a polymerase that can participate in the amplification reaction. In some cases, the first component comprises a primer that participates in the amplification reaction.

In another aspect, the disclosure provides a method for conducting a reduction reaction. The method comprises providing a reagent in a reaction volume, where the reagent comprises a first component coupled to a second component through a reducible linkage, and where the reaction volume includes a reducing agent that is in an inactive state such that the reducing agent is substantially unreactive with the reducible linkage; subjecting the reducing agent to an activation condition such that the reducing agent becomes reactive with the reducible linkage; and permitting the reducing agent to react with the reducible linkage to decouple the first component from the second component. In some cases, the method further comprises determining a sequence of at least a portion of a sequence of the amplification products.

In some cases, the reaction volume includes a template nucleic acid molecule and the first component comprises a primer. The method can further comprise, after permitting the reducing agent to react with the reducible linkage, subjecting the template nucleic acid molecule to an amplification reaction, using the first component, to yield nucleic acid molecules as amplification products of the template nucleic acid molecule. In some cases, the primer comprises a barcode sequence.

In some cases, the activation condition comprises an addition of thermal energy to the reaction volume. In some cases, the activation condition cleaves a covalent bond of the reducing agent. In some cases, the reducing agent, in its inactive state, is a stabilized reducing agent. In some cases, the stabilized reducing agent comprises a stabilized thiol containing compound. In some cases, the stabilized thiol containing compound comprises a sterically hindered thiol group.

In some cases, the stabilized thiol containing compound comprises a substituted dithiobutylamine (DTBA). In some cases, the substituted DTBA comprises:

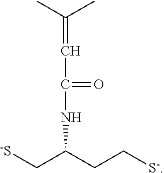

In some cases, the second component comprises a bead. In some cases, the first component comprises a nucleic acid molecule and the second component comprises a macromolecular matrix. In some cases, the macromolecular matrix comprises a polymer matrix. In some cases, the polymer matrix comprises a hydrogel. In some cases, the hydrogel comprises cross-linked polyacrylamide. In some cases, the cross-linked polyacrylamide is cross-linked by a reducible cross-linkage. In some cases, the reducible cross-linkage comprises a disulfide linkage. In some cases, after subjecting the reducing agent to the activation condition, the method further comprises cleaving the reducible cross-linkage.

In some cases, the reducible linkage comprises a disulfide linkage linking the nucleic acid molecule to the polymer matrix. In some cases, the nucleic acid molecule comprises a barcode sequence. In some cases, the reaction volume is provided in the aqueous interior of a droplet in a water-in-oil emulsion. In some cases, the reaction volume comprises an enzyme which can be, for example, a polymerase.

In another aspect, the disclosure provides a reducing agent comprising:

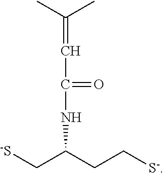

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 (panels A-D) provides chemical structures of example reducing agents;

FIG. 2 provides a graphic representation of dithiothreitol (DTT) oxidation (panel A), a graphic representation of dithiobutylamine (DTBA) oxidation (panel B) and a graphic representation of the functionality of an example substituted DTBA reducing agent described herein (panel C);

FIG. 3 (panels A-F) provides a schematic illustration of an example method for barcoding and amplification of nucleic acid fragments;

FIG. 4 provides a graphic representation of data obtained in experiments described in Example 1;

FIG. 5 (panels A and B) provides graphic representations of data obtained in experiments described in Example 2;

FIG. 6 (panels A, B and C) provides graphic representations of data obtained in experiments described in Example 3;

FIG. 7 (panels A, B and C) provides graphic representations of data obtained in experiments described in Example 4;

FIG. 8 (panels A, B and C) provides graphic representations of data obtained in experiments described in Example 5;

FIG. 9 (panels A, B and C) provides graphic representations of data obtained in experiments described in Example 6;

FIG. 10 (panels A and B) provides graphic representations of data obtained in experiments described in Example 7;

FIG. 11 provides a graphic representation of data obtained in experiments described in Example 8;

FIG. 12 provides a graphic representation of data obtained in experiments described in Example 9; and

FIG. 13 schematically depicts an example computer control system described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Stabilized Reducing Agents

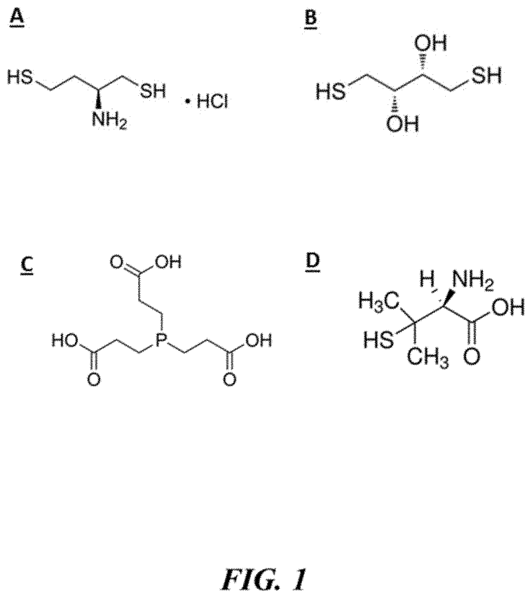

The present disclosure provides compositions that comprise stabilized reducing agents and uses of those compositions in a variety of different methods and processes. For a number of desired reactions, e.g., for analysis, processing or other uses, reducing agents are employed in order to initiate a desired reducing reaction and/or one or more reactions that make use of products of a reducing reaction, or prevent the initiation of undesired oxidation reactions. Examples of common reducing agents include (S)-2-Aminobutane-1,4-dithiol hydrochloride (e.g., see FIG. 1 panel A), dithiothreitol (DTT) (e.g., see FIG. 1 panel B) and Tris(2-carboxyethyl)phosphine (TCEP) (e.g., see FIG. 1 panel C).

The stabilized reducing agents described herein provide stability of a reactive reducing agent in one or more of a number of ways. A stabilized reducing reagent, as used herein, generally refers to a reducing agent that comprises structure and/or functionality that preserves reducing potential for desired reduction reactions by reducing or substantially inhibiting its reactivity in one or more undesired reactions, including e.g., reducing reactions, oxidation reactions, or other undesirable side reactions, volatilization, etc. In some cases, a stabilized reducing agent comprises one or more sterically-hindered functional groups having reducing activity in a reducing reaction. For example, in the case of a stabilized reducing agent comprising one or more reactive thiol groups, the thiol groups can be sterically hindered by one or more other functional groups in the molecule. Moreover, a stabilized reducing agent can also be stabilized by producing the reducing agent in an inactive state. In some cases, an active reducing agent can be transformed to an inactive state by substituting one or more functional groups of the reducing agent. Substitution can provide steric hindrance to a reducing agent's reducing functional group(s) that renders such groups less or not reactive. In some cases, a substituted form of a reducing agent can be converted an active state as is discussed below.

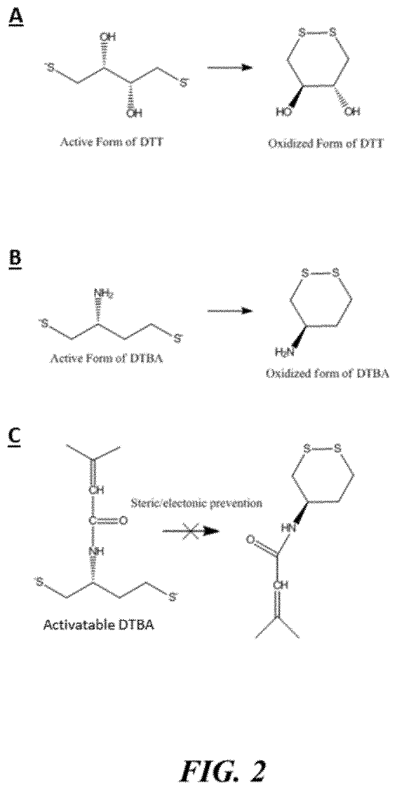

Moreover, a stabilized reducing reagent may be provided in a form that prevents oxidation under conventional storage conditions. Reducing agents can be prone to oxidation in that they react, including via an intramolecular reaction, to form oxidation products. For example, as shown in FIG. 2 (panel A), DTT can react with itself in an oxidation reaction (e.g., in the presence of air) to produce an oxidized form of DTT, having a ring structure. The oxidized form of DTT can be less active or ineffective when compared to its reduced form and, thus, such propensity to oxidation can limit the effectiveness of DTT as a reducing agent. In another example, as shown in FIG. 2 (panel B), dithiobutylamine (DTBA) can react with itself in an oxidation reaction to produce an oxidized form of DTBA, also having a ring structure. Similar to DTT, the oxidized form of DTBA can be less active or ineffective when compared to its reduced form and, thus, such propensity to oxidation can limit the effectiveness of DTBA as a reducing agent.

Additionally or alternatively, stabilized reducing reagents may be provided in a form that is less reactive with species where such reaction is a less desired side reaction to the reaction of interest, or where such reagents may be less volatile or otherwise transient. By providing stabilized reagents, one can be more certain that the original reactive concentration of a given reagent supplied will remain constant at the time of use, and within the useful concentration range. Alternatively, one may overdose a reaction with a given reagent, with potentially negative impacts on the reaction of interest, or one may precisely predict how much of the reactant will be present at the point the reaction occurs, under the particular conditions.

In some cases, for initiation of a desired reducing reaction, a threshold level of reducing agent can be needed. However, many reducing agents have relatively low stability when stored over time, due to for example, oxidation (e.g., DTT, DTBA as described above). As such, and as noted above, in order to ensure a sufficient amount of reducing agent can be delivered to the reaction of interest, it may require overdosing of the reaction, as one cannot be certain of the reducing power of older reagents. As noted above, overdosing a reaction with reducing agent may itself have significant negative impacts on the reaction or its constituent reactants. As such, it can be desirable to provide reducing agents that have relatively greater stability over time, so that one may more readily know the reducing power being applied to any given reaction.

For many cases, a reaction of interest can be highly sensitive to the amount of reactive reducing reagent present in the reaction mixture. As a result, minor fluctuations in the amount of reactive reducing agent within a reaction mixture can have substantial impact on the efficacy in the reaction, and in some cases, even have substantial negative impacts. Where reagents are transient in nature, these minor fluctuations may simply occur either with or without he understanding of the user. As a result, a reagent provided at a useful concentration may be outside of a useful concentration range by the time it is used. This can be problematic where reagents are intended to be shipped and stored for periods of time, e.g., as reagent kits for customer use. Moreover, in some cases, an unstable reducing agent may cause undesirable side reactions, which can result in unpredictable and often reduced amount of desired reaction products. In some cases, an unstable reducing agent may react with one or more non-desired species, which can affect reaction products generated from a reducing reaction and/or any downstream reducing reactions that make use of one or more products of the reducing reaction. Fluctuations in reducing agent concentration, as is discussed above, can further magnify the unpredictable/negative effects associated with an unstable reducing agent.

For example, a relatively less stable reducing agent, such as DTT or DTBA, can result in varied reaction performance (e.g., production of reaction products) in a reducing reaction and/or any downstream reaction that relies on the reducing activity of the reducing agent to make available one or more reaction components and/or conditions available for the downstream reaction due to variable concentrations of reducing agent in a reaction volume over time. Moreover, where inhibitory amounts of reducing are used (e.g., to compensate for losses in active reducing agent due to undesired oxidation), a relatively less stable reducing agent can exert inhibitory effects on one or more reaction components also resulting in variable and, perhaps, reduced reaction performance. For example, a relatively high concentration of reducing agent may inhibit an enzyme during a reaction, such as a polymerase in a nucleic acid amplification reaction. Accordingly, a stabilized reducing agent of the present disclosure can provide for more constant reaction performance (and, perhaps, higher product yield) in a reducing reaction and/or any downstream reaction that relies upon the reducing activity of the stabilized reducing agent to make one or more reaction components and/or conditions available for the downstream reaction.

The reducing activity of a stabilized reducing agent over the course of a reducing reaction and/or any downstream reaction that relies on the reducing activity of the stabilized reducing agent can be relatively constant. For example, the reducing activity of a stabilized reducing agent over the course of a reducing reaction and/or any downstream reaction that relies on the reducing activity of the stabilized reducing agent may vary by at most 50%, at most 40%, at most 35%, at most 30%, at most 25%, at most 20%, at most 15%, at most 10%, at most 9%, at most 8%, at most 7%, at most 6%, at most 5%, at most 4%, at most 3%, at most 2%, at most 1% or may vary by less over the course of the reaction(s). In another example, the reducing activity of the stabilized reducing agent may vary by at most 50%, at most 40%, at most 35%, at most 30%, at most 25%, at most 20%, at most 15%, at most 10%, at most 9%, at most 8%, at most 7%, at most 6%, at most 5%, at most 4%, at most 3%, at most 2%, at most 1%, over a time period of at most about 1 hour, 30 minutes, 20 minutes, 10 minutes, 5 minutes, 1 minutes, 30 seconds, 10 seconds, or 1 second, in some cases upon exposure to O₂. In some cases the reducing activity of a stabilized reducing agent over the course of a reducing reaction and/or any downstream reaction that relies on the reducing activity of the stabilized reducing agent may vary by at most 20% over the course of the reaction(s). In some cases the reducing activity of a stabilized reducing agent over the course of a reducing reaction and/or any downstream reaction that relies on the reducing activity of the stabilized reducing agent may vary by at most 10% over the course of the reaction(s). In some cases the reducing activity of a stabilized reducing agent over the course of a reducing reaction and/or any downstream reaction that relies on the reducing activity of the stabilized reducing agent may vary by at most 5% over the course of the reaction(s). In some cases the reducing activity of a stabilized reducing agent over the course of a reducing reaction and/or any downstream reaction that relies on the reducing activity of the stabilized reducing agent may vary by at most 1% over the course of the reaction(s).

A stabilized reducing agent may have substantially stable reducing activity. In some examples, in the absence of a reagent having a reducible linkage and in the presence of O₂ over a time period, a rate of decrease of a concentration of the stabilized reducing agent is less than a rate of decrease of a concentration of DTT, DTBA, or (S)-2-Aminobutane-1,4-dithiol hydrochloride over the time period. The stabilized reducing agent may have a lower oxidation rate as compared to DTT, DTBA, or (S)-2-Aminobutane-1,4-dithiol hydrochloride. The stabilized reducing agent may have higher selectivity for a reducible linkage (e.g., disulfide bond) as compared to DTT, DTBA, or (S)-2-Aminobutane-1,4-dithiol hydrochloride.

Penicillamine (e.g., see FIG. 1 panel D), including D-Penicillamine and L-Penicillamine, is an example of a stabilized reducing agent. Penicillamine can be a reactive reducing agent in both its D and L stereoisomer forms. Substitution of the α-carbon of penicillamine (e.g., with methyl groups) decreases the reactivity of the adjacent sulfur atom. Decreased reactivity of the thiol group permits penicillamine to function as a reducing agent generally without having the reactivity to participate in one or more side reactions that can inhibit a reaction dependent on the functionality of the reducing agent or another desired reaction. An additional example of a sterically-stabilized reducing agent is TCEP.

Furthermore, a stabilized reducing agent may function as an activatable reducing agent. In such cases, a stabilized reducing agent can be generally less reactive or not reactive in the context of a reduction reaction without activation. Moreover, a stabilized, activatable reducing agent can be activated by exposing the stabilized, activatable reducing agent (e.g., or a reaction volume comprising the activatable reducing agent) to one or more stimuli. Examples of such stimuli include thermal stimuli (e.g., addition or removal of thermal energy such as heat), chemical stimuli (e.g., contacting the stabilized, activatable reducing agent to one or more chemical activators) and physical stimuli (e.g., light). Upon exposure of the stabilized, activatable reducing agent to its appropriate stimulus or stimuli, the reducing agent can be converted to an active state. In some cases, exposure of a stabilized, activatable reducing agent to a stimulus cleaves one or more covalent bonds of the reducing agent such that it becomes active.

A stabilized, activatable reducing agent can be used to initiate and/or control the progress of a reduction reaction and, in some cases, one or more reactions that make use of one or more products of a reduction reaction. In some cases, a stabilized, activatable reducing agent can replace another activatable species in a reaction, with respect to reaction initiation and/or control. For example, in a nucleic acid amplification reaction, an activatable polymerase used in the reaction may be a hot-start polymerase, whereby an appropriate temperature is needed for suitable polymerase functionality. Upon exposing polymerase to the appropriate temperature, the nucleic acid amplification reaction is initiated. A stable, activatable reducing agent can be used as an alternative to a hot-start polymerase to initiate a nucleic acid amplification reaction. In such cases, a polymerase without hot-start dependent functionality can be used. Upon activation of the reducing agent, the reduction reaction and any downstream reactions can proceed. In some cases, both a reducing agent and another reagent can be activatable, e.g., hot start, in order to better control initiation and progression in a reaction of interest.

FIG. 2 (panel C) depicts an example of a stabilized, activatable reducing agent, in the form of a substituted DTBA (“Activatable DTBA” in FIG. 2 panel C). The substituted DTBA shown in FIG. 2 (panel C) can be generated by reacting DTBA with maleic anhydride. In its stabilized form, the substituted DTBA is generally incapable of intramolecular oxidation due to steric hindrance of its thiol groups as shown in FIG. 2 (panel C). In addition, steric hindrance of its thiol groups also decreases or inhibits its reactivity as a reducing agent. Upon exposure of the substituted DTBA to an appropriate temperature (e.g., via the addition of heat), citraconic acid can be cleaved from the substituted DTBA (e.g., via cleavage of the substituted DTBA's C—N covalent bond) to yield the active form of DTBA which can function in its capacity as a reducing agent. Stabilization of the DTBA as a substituted DTBA can prevent oxidation of DTBA to its oxidation product during storage and also permits its use in controlled reactions that are initiated by application of thermal stimulus to the DTBA.

Methods for Carrying Out Reactions with Use of Stabilized Reducing Reagents

Provided herein are methods of carrying out reactions. In one aspect, the disclosure provides a method for carrying out a reaction. The reaction can be conducted in a reaction volume that includes a reagent including a first component coupled to a second component through a reducible linkage. The reaction volume can also include a stabilized reducing reagent (e.g., that may or may not be active) that (e.g., upon activation) cleaves the reducible linkage and releases the first component from the second component. The released first component can then participate in the reaction. The first component can include one of a variety of different types of reagents or groups of reagents. For example, in certain cases, the first component may comprise a biological molecule, such as a protein, an enzyme, a peptide, small molecule, an antibody or antibody fragment or a nucleic acid, such as DNA, RNA, or oligo or polynucleotide portions of these. Moreover, any suitable stabilized reducing agent may be used, including stabilized thiol containing compounds (e.g., comprising a sterically hindered thiol groups such as penicillamine) and/or stabilized, activatable reducing agents (such as a substituted DTBA) as described elsewhere herein.

In another aspect, the disclosure provides a method for nucleic acid amplification. The method can include providing a reagent in a reaction volume, where the reagent comprises a first component coupled to a second component through a reducible linkage. The reaction volume can also include a template nucleic acid molecule and a stabilized reducing agent. The method can further include cleaving the reducible linkage with the aid of the stabilized reducing agent (e.g., thereby decoupling the first component from the second component) and subjecting the template nucleic acid molecule to an amplification reaction, using the first component, to yield nucleic acid molecules as amplification products of the template nucleic acid molecule. Any suitable stabilized reducing agent may be used, including stabilized thiol containing compounds (e.g., comprising a sterically hindered thiol group, such as, penicillamine) as described elsewhere herein. Moreover, the amplification reaction may proceed by cycling the temperature (e.g., thermocycling) of the reaction volume. In some cases, the method can further comprise determining at least a portion of a sequence of the amplification products as described elsewhere herein.

The reducing activity of the stabilized reducing agent with respect to the reducible linkage and over the course of providing the reaction volume, cleaving the reducible linkage and/or subjecting the template nucleic acid molecule to the amplification reaction can be relatively constant. Relatively constant reducing activity of the stabilized reducing agent can result in less variable amount of amplification products produced and/or higher yield. For example, the reducing activity of the stabilized reducing agent with respect to the reducible linkage and over the course of providing the reaction volume, cleaving the reducible linkage and/or subjecting the template nucleic acid molecule to the amplification reaction may vary by at most 50%, at most 40%, at most 35%, at most 30%, at most 25%, at most 20%, at most 15%, at most 10%, at most 9%, at most 8%, at most 7%, at most 6%, at most 5%, at most 4%, at most 3%, at most 2%, at most 1%, or may vary by less. In some cases, the reducing activity of the stabilized reducing agent with respect to the reducible linkage and over the course of providing the reaction volume, cleaving the reducible linkage and/or subjecting the template nucleic acid molecule to the amplification reaction may vary by at most 20%. In some cases, the reducing activity of the stabilized reducing agent with respect to the reducible linkage and over the course of providing the reaction volume, cleaving the reducible linkage and/or subjecting the template nucleic acid molecule to the amplification reaction may vary by at most 10%. In some cases, the reducing activity of the stabilized reducing agent with respect to the reducible linkage and over the course of providing the reaction volume, cleaving the reducible linkage and/or subjecting the template nucleic acid molecule to the amplification reaction may vary by at most 5%. In some cases, the reducing activity of the stabilized reducing agent with respect to the reducible linkage and over the course of providing the reaction volume, cleaving the reducible linkage and/or subjecting the template nucleic acid molecule to the amplification reaction may vary by at most 1%.

As described above, the stabilized reducing agent may have substantially stable reducing activity. In some examples, in the absence of the reagent and in the presence of O₂ over a time period, a rate of decrease of a concentration of the stabilized reducing agent is less than a rate of decrease of a concentration of DTT, DTBA, or (S)-2-Aminobutane-1,4-dithiol hydrochloride over the time period. The stabilized reducing agent may have a lower oxidation rate as compared to DTT, DTBA, or (S)-2-Aminobutane-1,4-dithiol hydrochloride. The stabilized reducing agent may have higher selectivity for a reducible linkage (e.g., disulfide bond) as compared to DTT, DTBA, or (S)-2-Aminobutane-1,4-dithiol hydrochloride.

In another aspect, the disclosure provides a method for conducting a reduction reaction. The method can include providing a reagent in a reaction volume, where the reagent comprises a first component coupled to a second component through a reducible linkage. The reaction volume can also include a reducing agent that is in an inactive state such that the reducing agent is substantially unreactive with the reducible linkage. The method can further include subjecting the reducing agent to an activation condition such that the reducing agent becomes reactive with the reducible linkage and permitting the reducing agent to react with the reducible linkage, thereby decoupling the first component from the second component. In general, the reducing agent in the reaction volume may be an activatable reducing agent in its inactive state, whereby the activation condition activates the activatable reducing agent such that it is reactive with the reducible linkage. In some cases, the reducing agent may comprise a stabilized, activatable reducing agent (e.g., a substituted DTBA) as described elsewhere herein.

In some cases, the activation condition can comprise applying a stimulus to the reaction volume and, thus, the reducing agent. As described elsewhere herein, examples of such stimuli include thermal stimuli (e.g., addition of thermal energy to the reaction volume), chemical stimuli and physical stimuli. In some cases, the activation condition comprises an addition of thermal energy to the reaction volume.

In some cases, the reaction volume may include a template nucleic acid and method can further comprise subjecting the template nucleic acid molecule to an amplification reaction, using the first component, to yield nucleic acid molecules as amplification products of the template nucleic acid molecule. Moreover, the method can also include determining a sequence of at least a portion of a sequence of the amplification products.

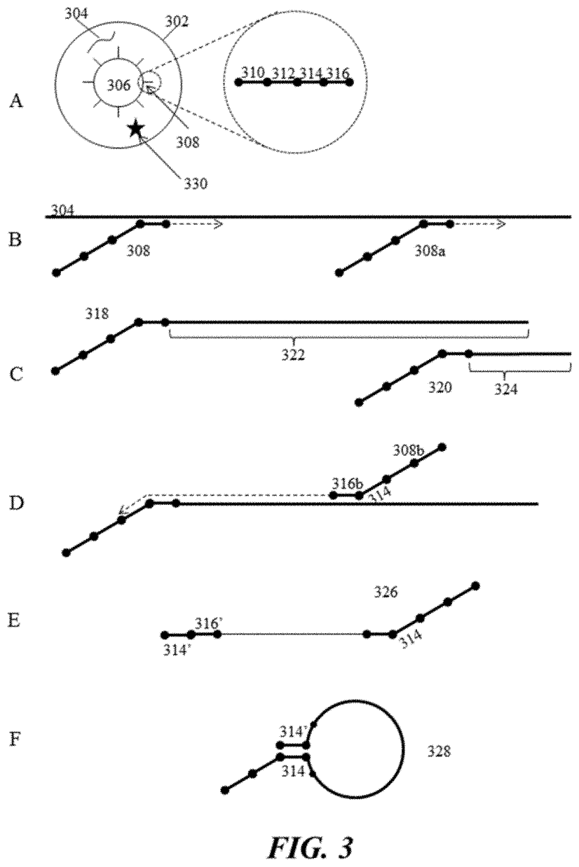

In various aspects, the first component may comprise a nucleic acid molecule such as, for example, a primer. Where a reaction volume comprises a template nucleic acid molecule, the primer can be used to amplify the template nucleic acid molecule to yield nucleic acid molecules as amplification products of the nucleic acid molecules. In some cases, the primer can comprise a barcode sequence that can be useful in nucleic acid sequencing, as is described elsewhere herein. Amplification of the template nucleic acid molecule with a primer comprising a nucleic acid barcode can generate amplification products that comprise the barcode sequence (e.g., see FIG. 3).

In various aspects, the second component can comprise a carrier component, which may include a carrier molecule and/or a carrier particle. Carrier components can comprise macromolecules (e.g. macromolecular matrices, such as polymeric matrices) or other components that can provide relative stability for a reagent in a given reaction volume and/or they may comprise active carrier components, e.g., that facilitate some additional activity, e.g., transport across cellular membranes and the like. In some cases, the carrier component may comprise a particle or bead component to which the first component is linked by a reducible linkage. For example, the first component may comprise a nucleic acid molecule and the second component may comprise a macromolecular matrix, where the macromolecular component may be included in a particle or bead. A wide range of particles have been described for use as carriers for reagents in general, and biological molecules in particular, including, e.g., organic and inorganic particles, such as agarose beads, silica beads, polyacrylamide beads, magnetic beads, ferromagnetic beads, and the like. Such beads can include active sites through which reagents may be bound or otherwise associated with the bead. As noted herein, that linkage can comprise a reducible linkage, e.g., a disulfide linkage, or the like.

In some cases, the carrier component may comprise a polymeric particle or bead that can comprise a polymer matrix of any suitable polymeric species' including a hydrogel. For example, the polymeric particle or bead may comprise a polymer matrix such as a matrix of cross-linked polyacrylamide (e.g., cross-linked linear polyacrylamide). The cross-linked polyacrylamide (e.g., cross-linked linear polyacrylamide) may be cross-linked by a reducible cross-linkage, such a disulfide linkage. Examples of polyacrylamide beads include those described in e.g., U.S. Patent Publication No. 2014/0378345 and U.S. Provisional Patent Application No. 62/163,238, filed May 18, 2015, the full disclosures of which are herein incorporated by reference in their entireties for all purposes. In some aspects, in addition to a reducible linkage between a first component and a second component, a reducing agent (e.g., stabilized reducing agent) may also cleave a reducible cross-linkage of the second component. Such cleavage can further aid in decoupling the first component from the second component. In one example, the second component may be a polyacrylamide gel bead comprising a cystamine cross-linkage. Upon exposure of the particle to a reducing agent (e.g., a stabilized reducing agent, an activated reducing agent), the disulfide bonds of the cystamine are broken and the particle can be degraded into its lower-order polymeric components.

In various aspects, the reaction volume may comprise an enzyme that participates in a reaction of the reaction volume. A stabilized reducing agent may prevent inhibitory side reactions of an enzyme with the reducing agent, including at relatively high concentrations of the reducing agent. In some cases, a reducing agent may inhibit an enzyme by reducing one or more disulfide linkages of the enzyme. For example, DTT can reduce the disulfide bonds of a polymerase, such that the disulfide bonds are cleaved into separate free thiol groups. Depending upon the particular enzyme, such a side reaction may be undesirable. A stabilized reducing agent, such as penicillamine, may reduce a disulfide bond of an enzyme by forming a disulfide bond with the enzyme, thus generating only a single free thiol, rather than a pair of thiols. Such functionality of the stabilized reducing agent can result in less variability/less inhibition in enzyme performance and, thus, less variability in and higher downstream product yields. Where a nucleic acid amplification reaction takes place, the enzyme may be a polymerase that participates in the nucleic acid amplification reaction. Non-limiting examples of polymerases include native and modified DNA polymerases, such as Taq polymerase, mutant proof reading polymerase, archeal polymerase, such as 9 degrees north, Pfu, Deep Vent, and exonuclease deficient versions of these enzymes, phi29 polymerase, Klenow, and the like.

Where a reaction includes a nucleic acid amplification reaction, such as amplification of a template nucleic acid, a reaction volume can include components suitable for a primer extension reaction (i.e., primers, one or more polymerase, dNTPs, and the like) that takes place as part of an amplification reaction. Examples of amplification reactions that may be completed include including polymerase chain reaction (PCR), digital PCR, reverse-transcription PCR, multiplex PCR, nested PCR, overlap-extension PCR, quantitative PCR, multiple displacement amplification (MDA), or ligase chain reaction (LCR) and amplification in droplets (e.g., digital PCR). Additional examples of amplification reactions are provided by U.S. Patent Publication No. 2014/0378345, the full disclosure of which is incorporated herein by reference in its entirety for all purposes.

Various aspects include a reaction volume which may be provided in any suitable reaction vessel. Non-limiting examples of reaction vessels that can contain a reaction volume include tubes (e.g., centrifuge tubes, micro-centrifuge tubes, vials, test tubes, etc.), wells, wells of a multi-well plate (e.g., microplate), beakers, flasks, microcapsules and micelles. Another example type of reaction vessel is a droplet of an emulsion. An emulsion (e.g., a water-in-oil emulsion, an oil-in-water emulsion) can provide a plurality of droplets, each comprising a reaction volume. As a result, emulsion and droplet technology can permit a large number of simultaneous reaction volumes (e.g., individual droplets each having a reaction volume) in a single medium (e.g., the emulsion).

Droplets can be formed when two or more immiscible liquids are mixed such as, for example, water and oil. An example of a mixture comprising two or more immiscible liquids is an emulsion, such as a water-in-oil emulsion. The first liquid, which is dispersed in globules, can be referred to as a discontinuous phase, whereas the second liquid, in which the globules are dispersed, can be referred to as a continuous phase or dispersion medium. In some examples, the continuous phase can be a hydrophobic fluid, such as an oil, and the discontinuous phase can be an aqueous phase solution. Such a mixture can be considered a water-in-oil emulsion, wherein aqueous droplets are dispersed in an oil continuous phase. In other cases, an emulsion may be an oil-in-water emulsion. In such an emulsion, the discontinuous phase can be a hydrophobic solution (e.g., oil) and the continuous phase can be an aqueous solution, wherein droplets of oil are dispersed in an aqueous phase. In some examples, the emulsion may comprise a multiple emulsion. Multiple emulsions can comprise larger fluidic droplets that encompass one or more smaller droplets (i.e., a droplet within a droplet). Multiple emulsions can contain one, two, three, four, or more nested fluids generating increasingly complex droplets within droplets.

An oil of an emulsion may be selected based upon chemical properties, such as, among others molecular structure, content, solvating strength, viscosity, boiling point, thermal expansion coefficient, oil-in-water solubility, water-in-oil solubility, dielectric constant, polarity, water-in-oil surface tension, and/or oil-in-water surface tension. Examples of oils useful in an emulsion (e.g., a water-in-oil emulsion) include, without limitation, fluorinated oils, non-fluorinated oils, alkanes, oils comprising trifluoroacetic acid, oils comprising hexafluoroisopropanol, Krytox oils (e.g., oils comprising hexafluoropropylene epoxide and/or polymers thereof), oil comprising polyhexafluoropropylene oxide and/or polymers thereof, Krytox GPL oils, oils comprising perfluoropolyether, oils comprising perfluoroalkylether, oils comprising perfluoropolyalkylether, Solvay Galden oils, and oils including hydrofluoroethers (e.g., HFE-7500, HFE-7100, HFE-7200, HFE-7600).

An emulsion may further comprise a surfactant. The surfactant may be a fluorosurfactant. Surfactants may stabilize droplets in a continuous phase. Examples of fluorosurfactants useful for stabilizing droplets in fluorinated and other oils are described in detail in U.S. Patent Publication No. 2010/0105112, the full disclosure of which is hereby incorporated by reference in its entirety. In some examples, a water-in-oil emulsion may comprise one or more of the oils described herein having one or more surfactants (e.g., fluorosurfactants), wherein aqueous droplets are dispersed in the oil(s).

Droplets may be formed by a variety of methods. Emulsion systems for creating stable droplets in non-aqueous or oil continuous phases are described in detail in, e.g., U.S. Patent Publication No. 2010/0105112. In some cases, microfluidic channel networks can be used for generating droplets. Examples of such microfluidic devices include those described in detail in U.S. patent application Ser. No. 14/682,952 filed Apr. 9, 2015, the full disclosure of which is incorporated herein by reference in its entirety for all purposes. Droplets may be formed with a regular periodicity or may be formed with an irregular periodicity. In some aspects, the size and/or shape of the droplet may be determined by the size and shape of a channel in which the droplet is formed.

In some examples, droplets may generally be generated by flowing an aqueous stream (e.g., comprising the components of a reaction volume such as a reagent and reducing agent (stabilized reducing agent, etc.) into a junction of two or more channels of a microfluidic system into which is also flowing a non-aqueous stream of fluid, e.g., a fluorinated oil, such that aqueous droplets are created within the flowing stream non-aqueous fluid. The droplet contents comprise aqueous interiors comprising the reaction volumes. The relative amount of species within a droplet may be adjusted by controlling a variety of different parameters of the system, including, for example, the concentration of species in the aqueous stream, the flow rate of the aqueous stream and/or the non-aqueous stream, and the like.

Droplets may have overall volumes that are less than 1000 pL, less than 900 pL, less than 800 pL, less than 700 pL, less than 600 pL, less than 500 pL, less than 400 pL, less than 300 pL, less than 200 pL, less than 100 pL, less than 50 pL, less than 20 pL, less than 10 pL, or even less than 1 pL. Droplets may be monodisperse (i.e., substantially uniform in size) or polydisperse (i.e., substantially non-uniform in size). A plurality of droplets may be generated.

An emulsion may comprise a varied number of droplets depending upon the particular emulsion. For example, an emulsion may comprise at least 10 droplets, at least 50 droplets, at least 100 droplets, at least 500 droplets, at least 1000 droplets, at least 5000 droplets, at least 10,000 droplets, at least 50,000 droplets, at least 100,000 droplets, at least 500,000 droplets, at least 1,000,000 droplets, at least 5,000,000 droplets, at least 10,000,000 droplets, at least 50,000,000 droplets, at least 100,000,000 droplets and upwards.

### Stabilized Reducing Agents for Use in Preparation of Nucleic Acid Sequencing Libraries

Stabilized reducing agents described herein can be useful in sample preparation for nucleic acid sequencing. Such reducing agents can be used to control nucleic acid amplification reactions that can generate sequencing libraries. In some cases, a library of nucleic acid molecules can be generated, wherein the library comprises a plurality of droplets or other type of partitions comprising the nucleic acid molecules. Examples of preparing a library of nucleic acid molecules in partitions are provided in detail in e.g., U.S. Patent Publication No. 2014/0378345, U.S. Provisional Patent Application No. 62/017,808, filed Jun. 26, 2014 and U.S. Provisional Patent Application 62/102,420, filed Jan. 12, 2015, the full disclosures of which are incorporated by reference in their entireties for all purposes). Where the library of nucleic acid molecules comprises a plurality of droplets having the nucleic acid molecules, the plurality of droplets can be destabilized, thereby releasing the nucleic acid molecules from the plurality of droplets into a common pool. The released nucleic acid molecules (e.g., target nucleic acid molecules) can be recovered/purified from the common pool. Purification methods suitable for purifying the contents of droplets, including nucleic acids, are provided in detail in U.S. Provisional Patent Application No. 61/119,930, filed on Feb. 24, 2015, the full disclosure of which is incorporated herein by reference in its entirety for all purposes. The purified nucleic acid molecules can optionally be subject to further processing as described elsewhere herein and subject to sequencing, whereby the sequences of at least a subset of the purified nucleic acid molecules (or further processed purified nucleic acid molecules) can be determined. Sequencing may be performed via any suitable type of sequencing platform including example platforms described elsewhere herein.

FIG. 3 shows an example of an amplification reaction that can be performed in a droplet (e.g., having a reaction volume) and can be useful for generating a nucleic acid sequencing library in a plurality of droplets. In this example, oligonucleotides that include a barcode sequence are co-partitioned in a droplet 302 in an emulsion, along with a sample nucleic acid 304. The oligonucleotides 308 may be coupled to a bead 306 through a reducible linkage (e.g., disulfide linkages), which bead can be co-partitioned with the sample nucleic acid 304, as shown in panel A. The bead 306 can also comprise a polymeric matrix of cross-linked polyacrylamide, cross-linked via a reducible linkage (e.g., disulfide linkage), which may or may not be the same reducible linkage that links the oligonucleotides 308 to the bead 306. The oligonucleotides 308 include a barcode sequence 312, in addition to one or more functional sequences, e.g., sequences 310, 314 and 316. For example, oligonucleotide 308 is shown as comprising barcode sequence 312, as well as sequence 310 that may function as an attachment or immobilization sequence for a given sequencing system, e.g., a P5 sequence used for attachment in flow cells of an Illumina Hiseq or Miseq system. As shown, the oligonucleotides 308 also include a primer sequence 316, which may include a random or targeted N-mer for priming replication of portions of the sample nucleic acid 304. Also included within oligonucleotide 308 can be a sequence 314 which may provide a sequencing priming region, such as a “read1” or R1 priming region, that may be used to prime polymerase mediated, template directed sequencing by synthesis reactions in sequencing systems. In many cases, the barcode sequence 312, immobilization sequence 310 and R1 sequence 314 may be common to all of the oligonucleotides attached to a given bead. The primer sequence 316 may vary for random N-mer primers, or may be common to the oligonucleotides on a given bead for certain targeted applications.

Droplet 302 can also include a stabilized reducing agent 330. In some cases, the stabilized reducing agent 330 may be in an active state (e.g., penicillamine) such that it can reduce reducible linkages (e.g., disulfide linkages) between the oligonucleotides 308 and bead 306 and/or any reducible cross-linkages (e.g., disulfide linkages) of bead 306. The coupled oligonucleotides 308 and bead 306 can be decoupled upon cleavage of their reducible linkages by stabilized reducing agent 330. The decoupled oligonucleotides can then participate as primers in an amplification reaction that amplifies sample nucleic acid 304 (e.g., FIG. 3 panels B-F, as described below).

The reducing activity of the stabilized reducing agent 330 can be relatively constant over the course of reducing reducible linkages and/or conducting the amplification reaction. Relatively constant reducing activity of the stabilized reducing agent can result in less variability in and higher amplification product 328 yield. For example, the reducing activity of the stabilized reducing agent 330 over the course of decoupling oligonucleotides 308 from bead 306 and/or amplifying the sample nucleic acid 304 in the amplification reaction may vary by at most 50%, at most 40%, at most 35%, at most 30%, at most 25%, at most 20%, at most 15%, at most 10%, at most 9%, at most 8%, at most 7%, at most 6%, at most 5%, at most 4%, at most 3%, at most 2%, at most 1%, or may vary by less. In some cases, the reducing activity of the stabilized reducing agent 330 over the course of decoupling oligonucleotides 308 from bead 306 and/or amplifying the sample nucleic acid 304 in the amplification reaction may vary by at most 20%. In some cases, the reducing activity of the stabilized reducing agent 330 over the course of decoupling oligonucleotides 308 from bead 306 and/or amplifying the sample nucleic acid 304 in the amplification reaction may vary by at most 10%. In some cases, the reducing activity of the stabilized reducing agent 330 over the course of decoupling oligonucleotides 308 from bead 306 and/or amplifying the sample nucleic acid 304 in the amplification reaction may vary by at most 5%. In some cases, the reducing activity of the stabilized reducing agent 330 over the course of decoupling oligonucleotides 308 from bead 306 and/or amplifying the sample nucleic acid 304 in the amplification reaction may vary by at most 1%.

In some cases, stabilized reducing agent 330 may be a stabilized, activatable reducing agent initially present in droplet 302 in an inactive state. For example, stabilized reducing agent 330 may be a substituted DTBA such as that shown FIG. 2 (panel C). The droplet 302 can be exposed to a thermal stimulus (e.g., an addition of thermal energy such as heat to the droplet 302) in order to cleave citraconic acid from the substituted DTBA and yield the active DTBA reducing agent. Where the reducible linkages between oligonucleotides 308 and bead 306 are disulfide linkages, the active DTBA reducing agent can then reduce reducible linkages between the oligonucleotides 308 and bead 306. If bead 306 also comprises disulfide cross-linkages, active DTBA can also reduce these linkages. The coupled oligonucleotides 308 and bead 306 can be decoupled upon cleavage of their reducible linkages by the stabilized reducing agent. The decoupled oligonucleotides can then participate as primers in an amplification reaction that amplifies sample nucleic acid 304 (e.g., FIG. 3 panels B-F, as described below).

As shown in FIG. 3, control and initiation of the amplification reaction shown in panels B-F can be exerted via activation of the stabilized reducing agent 330 in panel A. Amplification as shown in panels B-F of FIG. 3 generally does not commence until oligonucleotides 308 are released from bead 306. Moreover, the use of a stabilized, activatable reducing agent can reduce or eliminate any need to use another activatable component to initiate/control the amplification reactions. For example, use of the substituted DTBA shown in FIG. 2 panel C can function as an activatable substitute to a hot-start polymerase that can also be used to initiate/control the amplification reactions.

Following release of the oligonucleotides 308 to bead 306, the amplification reaction can proceed. Based upon the presence of primer sequence 316, the decoupled oligonucleotides 308 are able to prime the sample nucleic acid as shown in panel B, which allows for extension of the oligonucleotides 308 and 308a using polymerase enzymes (e.g., Deep Vent polymerase, 9 degrees North polymerase) and other extension reagents also co-partitioned with the bead 306 and sample nucleic acid 304. As shown in panel C, following extension of the oligonucleotides that, for random N-mer primers, may anneal to multiple different regions of the sample nucleic acid 304; multiple overlapping complements or fragments of the nucleic acid are created, e.g., fragments 318 and 320. Although including sequence portions that are complementary to portions of sample nucleic acid, e.g., sequences 322 and 324, these constructs are generally referred to herein as comprising fragments of the sample nucleic acid 304, having the attached barcode sequences. As can be appreciated, the replicated portions of the template sequences as described above are often referred to herein as “fragments” of that template sequence. Notwithstanding the foregoing, however, the term “fragment” encompasses any representation of a portion of the originating nucleic acid sequence, e.g., a template or sample nucleic acid, including those created by other mechanisms of providing portions of the template sequence, such as actual fragmentation of a given molecule of sequence, e.g., through enzymatic, chemical or mechanical fragmentation. In some cases, however, fragments of a template or sample nucleic acid sequence can denote replicated portions of the underlying sequence or complements thereof.

The barcoded nucleic acid fragments may then be subjected to characterization, e.g., through sequence analysis, or they may be further amplified in the process, as shown in panel D. For example, additional oligonucleotides, e.g., oligonucleotide 308b, also released from bead 306, may prime the fragments 318 and 320. Again, based upon the presence of the random N-mer primer 316b in oligonucleotide 308b (which in many cases may be different from other random N-mers in a given droplet, e.g., primer sequence 316), the oligonucleotide anneals with the fragment 318, and can be extended to create a complement 326 to at least a portion of fragment 318 which includes sequence 322, that comprises a duplicate of a portion of the sample nucleic acid sequence. Extension of the oligonucleotide 308b continues until it has replicated through the oligonucleotide portion 308 of fragment 318. As noted elsewhere herein, and as illustrated in panel D, the oligonucleotides may be configured to prompt a stop in the replication by the polymerase at a desired point, e.g., after replicating through sequences 316 and 314 of oligonucleotide 308 that is included within fragment 318. This may be accomplished by different methods, including, for example, the incorporation of different nucleotides and/or nucleotide analogues that are not capable of being processed by the polymerase enzyme used. For example, this may include the inclusion of uracil containing nucleotides within the sequence region 312 to prevent a non-uracil tolerant polymerase to cease replication of that region. As a result a fragment 326 can be created that includes the full-length oligonucleotide 308b at one end, including the barcode sequence 312, the attachment sequence 310, the R1 primer region 314, and the random N-mer sequence 316b. At the other end of the sequence may be included the complement 316′ to the random N-mer of the first oligonucleotide 308, as well as a complement to all or a portion of the R1 sequence, shown as sequence 314′. The R1 sequence 314 and its complement 314′ are then able to hybridize together to form barcoded nucleic acid molecules in a partial hairpin structure 328. As can be appreciated because the random N-mers differ among different oligonucleotides, these sequences and their complements may not be expected to participate in hairpin formation, e.g., sequence 316′, which is the complement to random N-mer 316, may not be expected to be complementary to random N-mer sequence 316b. This may not be the case for other applications, e.g., targeted primers, where the N-mers may be common among oligonucleotides within a given droplet. By forming these partial hairpin structures 328, it allows for the removal of first level duplicates of the sample sequence from further replication, e.g., preventing iterative copying of copies. The partial hairpin structure also provides a useful structure for subsequent processing of the created fragments, e.g., fragment 326.

As can be appreciated, the example amplification scheme depicted in FIG. 3 may be completed in any suitable type of partition, including non-droplet partitions, such as microcapsules, wells (e.g., microwells), polymeric capsules, microreactors, micelles, etc.

Additional examples of amplification reactions that can be performed in droplets or other types of partitions, including amplification reactions that can be used to generate nucleic acid libraries for sequencing, are provided in U.S. Provisional Patent Application No. 62/102,420, filed Jan. 12, 2015, which is incorporated herein by reference in its entirety for all purposes.

An amplification process used to generate barcoded nucleic acid molecules, such as the example method depicted in FIG. 3 can be conducted in parallel for a plurality of droplets. Fragments from multiple different droplets can be pooled (e.g., by collecting droplets and destabilizing the emulsion as described elsewhere herein) to generate a sequencing library for sequencing on high throughput sequencers. Because each fragment is coded as to its droplet of origin, the sequence of that fragment may be attributed back to its origin based upon the presence of the barcode.

Releasing the contents of a droplet can encompass any method by which the contents of a droplet are liberated. Non-limiting examples of release methods include breaking the surface of a droplet, making the droplet porous such that the contents can diffuse out of the droplet, and destabilizing the emulsion in which a droplet is present. An emulsion can be mixed with a destabilization agent that causes the droplets to destabilize and to coalesce. A destabilization agent can be any agent that induces droplets of an emulsion to coalesce with one another. The destabilization agent may be present at an amount effective to induce coalescence, which may be selected based, for example, on the volume of the emulsion, the volume of carrier fluid in the emulsion, and/or the total volume of droplets, among others. The amount also or alternatively may be selected, based, for example, on the type of continuous phase fluid, amount and type of surfactant in each phase, etc. In some cases, a destabilization agent may be a weak surfactant. Without wishing to be bound by theory, a weak surfactant can compete with droplet surfactant at the oil/aqueous interface causing an emulsion to collapse. In some cases, the destabilization agent can be perfluorooctanol (PFO), however, other fluorous compounds with a small hydrophilic group may be used. Other examples of destabilization agents include one or more halogen-substituted hydrocarbons. In some cases, the destabilization agent may be predominantly or at least substantially composed of one or more halogen-substituted hydrocarbons. Additional examples of destabilization agents are provided in U.S. Patent Publication No. 2013/018970 and U.S. Provisional Patent Application No. 62/119,930, filed Feb. 24, 2015, the full disclosures of which is incorporated herein by reference for all purposes.

Coalescence of the droplets can result in the generation of a pooled mixture (e.g., a common pool) comprising the contents of the droplets (e.g., contents of reaction volumes contained within the droplets). Where the droplets are aqueous droplets in a water-in-oil emulsion, the pooled mixture may comprise an aqueous mixture having the contents of a plurality of reaction volumes including barcoded nucleic acid molecules generated in the droplets. Continuous phase (e.g., oil) material in which the droplets were originally dispersed, surfactants in the continuous phase, and/or any destabilization agent can be removed via purification of the nucleic acids in the pooled mixture. Examples of purification methods suitable for purifying nucleic acid molecules from droplets are described in detail in U.S. Provisional Patent Application No. 62/119,930, filed Feb. 24, 2015, the full disclosure of which is incorporated herein by reference for all purposes.

Barcoded nucleic acid molecules that have been recovered from droplets and purified can subject to further processing and/or analysis. For example, barcoded nucleic acid molecules recovered from droplets may be further amplified, such as in a PCR reaction or other type of amplification reaction. Such amplification may be useful where recovered, barcoded nucleic acid molecules are initially present in low amounts and greater copy numbers are helpful for downstream sequencing analysis. Moreover, such amplification reactions may also be completed to add one or more additional sequences (e.g., append additional nucleotides) to the recovered barcoded nucleic acid molecules. Such additional sequences can result in the generation of larger nucleic acid molecules and the one or more added sequences may be one or more functional sequences. Non-limiting examples of such functional sequences include a tag, an additional barcode sequence, an adapter sequence for sequence compatibility with a sequencing instrument/protocol (e.g., P5, P7 Illumina adaptor sequences), a sequencing primer binding site, a sample index sequence, etc. Examples of adding additional sequences to nucleic acid molecules via an amplification reaction (including a bulk amplification reactions) are provided in U.S. Patent Publication No. 2014/0378345 and U.S. Provisional Patent Application No. 62/102,420, filed Jan. 12, 2015, the full disclosures of which is incorporated herein by reference in its entirety for all purposes.

In some cases, one or more additional sequences may be added to barcoded nucleic acid molecules via a ligation process to generate larger barcoded nucleic acid molecules that can then be sequenced. In some cases, the target nucleic acid molecules may be subject to a shearing process in order to generate one or more ends of the target nucleic acid molecules that are suitable for ligation with an additional nucleic acid sequence. The additional nucleic acid sequence may comprise one or more of any of the functional sequences described herein. Examples of shearing and ligation methods that can be used for adding additional sequences to nucleic acid molecules are provided in detail in U.S. Provisional Patent Application No. 62/102,420, filed Jan. 12, 2015, the full disclosure of which is incorporated by reference in its entirety for all purposes. Upon addition of the additional sequence(s) to the barcoded nucleic acid molecules, the larger sequences that are generated can be amplified to provide greater copy numbers. Shearing, ligation and any subsequent amplification can be performed in bulk.

Barcoded nucleic acid molecules (that may or may not be further processed and/or purified) or barcoded nucleic acid molecules to which one or more additional sequences have been appended may be subject to nucleic acid sequencing, whereby a sequence of the barcoded nucleic acid molecules or larger barcoded nucleic acid molecules can be determined. The addition of additional functional sequences to barcoded nucleic acid molecules may be useful in preparing the barcoded nucleic acid molecules for sequencing. Barcoded nucleic acid molecules may be prepared for any suitable sequencing platform and sequenced, with appropriate functional sequences added to barcoded nucleic acid molecules depending on the particular platform utilized. Sequencing may be performed via any suitable type of sequencing platform, with non-limiting examples that include Illumina, Ion Torrent, Pacific Biosciences SMRT, Roche 454 sequencing, SOLiD sequencing, etc. As can be appreciated, sequences obtained from nucleic acid molecules can be assembled into larger sequences from which the sequence of the nucleic acid molecules originated. In general, sequencing platforms make use of one or more algorithms to interpret sequencing data and reconstruct larger sequences from sequenced determined for shorter nucleic acids, including the barcoded nucleic acid molecules described herein (e.g., 328 in FIG. 3). Examples of sequence assembly processes are provided in greater detail in U.S. Provisional Patent Application No. 62/017,589, filed on Jun. 26, 2014, the full disclosure of which is herein incorporated by reference for all purposes.

As can be appreciated, use of a stabilized reducing agent as part of a reaction scheme including amplification of nucleic acid molecules to be sequenced can improve the performance of sequencing. The generation of consistent (and, perhaps, higher) amount of amplification products can improve the quality of downstream sequencing data that is obtained and/or any subsequent analysis of such data. In some cases, use of a stabilized reducing agent to initiate an amplification reaction, such the example method shown in FIG. 3, can reduce the rate of chimera observed in downstream sequencing data.

### Kits

In another aspect, the present disclosure provides a kit comprising one or more reagents and/or vessels for conducting a reduction reaction, perhaps in conjunction with a nucleic acid amplification reaction. Accordingly, a kit may include one or more of the following: a stabilized reducing agent (including a type described herein such as penicillamine), an activatable reducing agent (e.g., including a stabilized, activatable reducing agent such as a substituted DTBA), beads (e.g., polymeric beads, such as polyacrylamide beads) comprising oligonucleotide barcodes, and reagents for conducting nucleic acid amplification (e.g., one or more primers, one or more polymerases, dNTPs, co-factors, etc.). In some cases, a kit may comprise reagents suitable for generating an emulsion. Non-limiting examples of such reagents include a continuous phase (e.g., oil) and an aqueous phase (e.g., a buffer). A kit may also comprise packaging (i.e., a box). The reagents and the device may be packaged into a single kit. Alternatively, the reagents and the device may be packaged separately. The kits may further comprise instructions for usage of the kit. These instructions may be in the form of a paper document or booklet contained within the packaging of the kit. Alternatively, the instructions may be provided electronically (i.e., on the Internet).

### Computer Control Systems

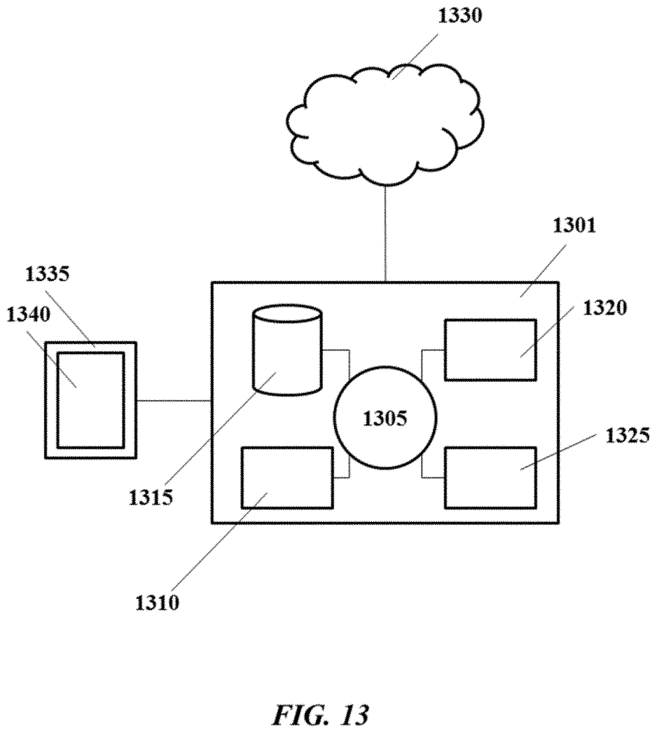

The present disclosure provides computer control systems that are programmed to implement methods of the disclosure. FIG. 13 shows a computer system 1301 that is programmed or otherwise configured to control and/or execute nucleic acid amplification reactions and/or reduction reactions described herein. The computer system 1301 can, for example, regulate various aspects of reaction parameters, including reagent amounts, reaction conditions (e.g., temperature, pressure, humidity), fluid handling devices, reaction equipment, etc. of the present disclosure. The computer system 1301 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, reaction reagent amounts, reaction parameters (e.g., temperature, pressure, stirring rates, time, etc.) displaying the results of a reaction (e.g., product yield, product quality, etc.), raw data measured from a reaction, analyzed or processed data measured for a reaction, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1305. The algorithm can, for example, execute a nucleic acid amplification reaction and/or reduction reaction described herein and/or obtain and analyze data obtained from such reactions.

### EXAMPLES

### Example 1: Effect of DTT and Polymerase Preparation on Amplification Reaction Performance

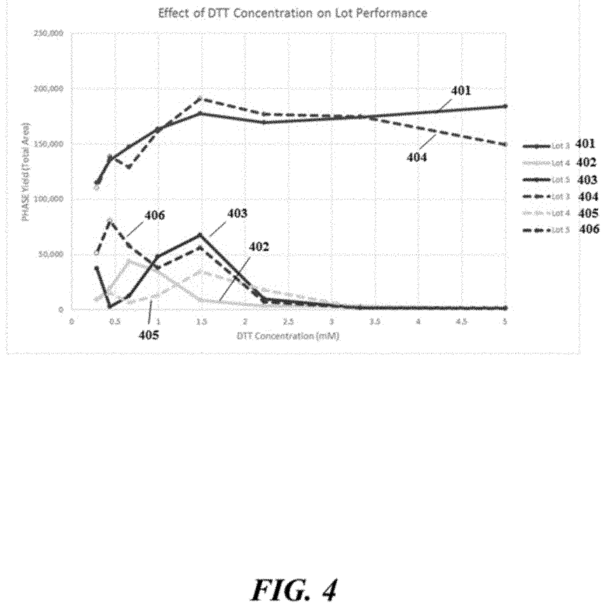

Three sets of parallel amplification experiments were conducted. Each experimental set included replicate reaction volumes, with each reaction volume comprising polyacrylamide gel beads linked (via disulfide linkages) to oligonucleotides comprising barcode and priming sequences similar to oligonucleotides 308 shown in FIG. 3, sample nucleic acid molecules, a 9 degrees north polymerase and DTT at an initial concentration all in a reaction buffer. Each set of experiments included a different 9 degrees north polymerase preparation (“Lot 3”, “Lot 4” and “Lot 5” as shown in FIG. 4) and reaction volumes in each experimental set varied in their initial concentration of DTT. Each reaction volume was subjected to thermal cycling to amplify the sample nucleic acid molecules in an amplification reaction similar to the example amplification method graphically depicted in FIG. 3. While partial hairpin containing amplification products were generated, amplification reactions were completed in bulk reaction volumes, rather than in droplets in an emulsion as shown in FIG. 3. During the amplification reactions, DTT reduced the disulfide linkages between oligonucleotides and beads, initiating the amplification reaction.

Amplification product yield versus initial reducing agent concentration for the various experimental sets are graphically depicted in FIG. 4. Experimental sets for Lot 4 (replicates 402 and 405 in FIG. 4) and Lot 5 (replicates 403 and 406) showed higher amounts of amplification products at generally lower initial concentrations of DTT, with substantially lower to no amount of amplification products at higher concentrations. Conversely, amplification products generated in the Lot 3 experimental set (replicates 401 and 404 in FIG. 4) were comparable or better than those in Lots 4 and 5 and were sustained across the range of initial DTT concentrations tested. Data indicate that reaction performance can vary with different polymerase preparation, when DTT is used as a reducing agent. Such differences may be attributable to different oxidation states of the enzymes across the various lots tested and/or the relatively unstable nature of DTT as described elsewhere herein.

### Example 2: Effect of Reducing Agent Type on Amplification Reaction Performance

Four sets of parallel amplification experiments were conducted. Each experimental set included replicate reaction volumes, with each reaction volume comprising polyacrylamide gel beads linked (via disulfide linkages) to oligonucleotides comprising barcode and priming sequences similar to oligonucleotides 308 shown in FIG. 3, sample nucleic acid molecules, a 9 degrees north polymerase and a reducing agent all in a reaction buffer. Each set of experiments included a different reducing agent (DTT, TCEP, Penicillamine and (S)-2-Aminobutane-1,4-dithiol hydrochloride as shown in FIG. 4) and reaction volumes in each experimental set varied in their initial concentration of reducing agent. Each reaction volume was subjected to thermal cycling to amplify the sample nucleic acid molecules in an amplification reaction similar to the example amplification method graphically depicted in FIG. 3. While partial hairpin containing amplification products were generated, amplification reactions were completed in bulk reaction volumes, rather than in droplets in an emulsion as shown in FIG. 3. During the amplification reactions, the appropriate reducing reagent reduced the disulfide linkages between oligonucleotides and beads, initiating the amplification reaction.

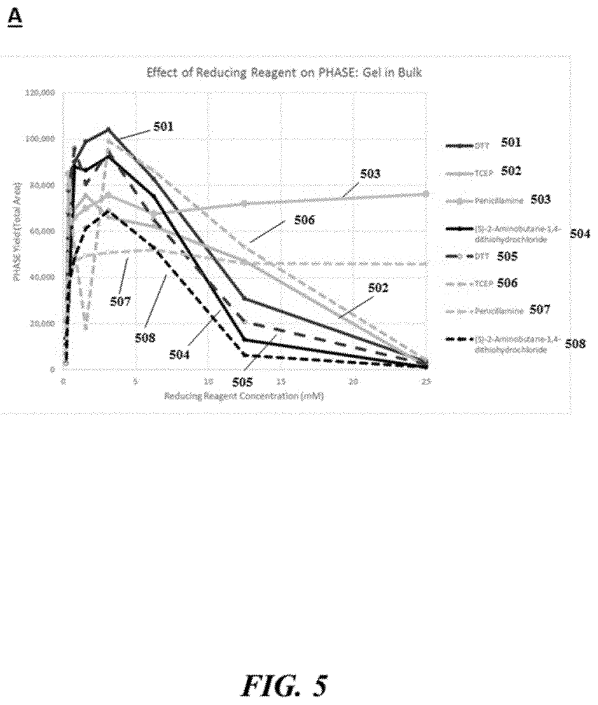

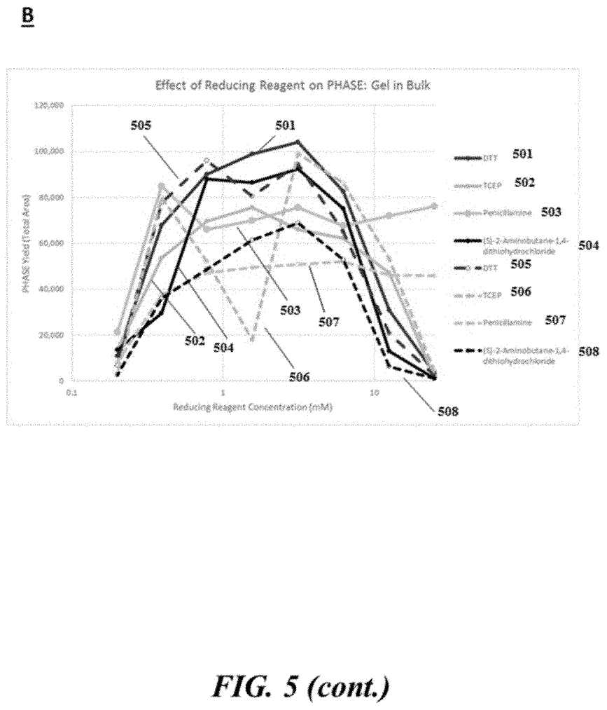

Amplification product yield for the various experimental sets are graphically depicted in FIG. 5. FIG. 5 (panel A) graphically depicts amplification yield with varied initial reducing reagent concentration on a linear x-axis, whereas FIG. 5 (panel B) depicts the same data using a logarithmic x-axis. Experimental sets for DTT (replicates 501 and 505 in FIG. 5), TCEP (replicates 502 and 506) and (S)-2-Aminobutane-1,4-dithiol hydrochloride (replicates 504 and 508) showed higher amounts of amplification products at generally lower initial concentrations of reducing agent, with substantially lower amounts of amplification products at higher concentrations.

Conversely, amplification products generated in the Penicillamine experimental set (replicates 503 and 507 in FIG. 5) varied some with initial concentration of reducing agent, however produced amplification products in amounts comparable to or better than the other reducing agents and, unlike the other reducing agents, across the range of initial Penicillamine concentrations tested. Data indicate that Penicillamine can yield amplification products in relatively similar amount across a broad range of initial reducing agent concentration when compared to other reducing reagents such as DTT, TCEP and (S)-2-Aminobutane-1,4-dithiol hydrochloride. Such differences may be attributable the stabilized nature (e.g., via its sterically hindered thiol group) of Penicillamine, as described elsewhere herein.

### Example 3: Effect of Reducing Agent Type and Amplification Product Yield

Three sets of experiments were performed to compare the performance of DTT and Penicillamine in generating amplification products in an amplification reaction. In a first set of experiments, the set included replicate reaction volumes, with each reaction volume comprising polyacrylamide gel beads linked (via disulfide linkages) to oligonucleotides comprising barcode and priming sequences similar to oligonucleotides 308 shown in FIG. 3, sample nucleic acid molecules, a 9 degrees north polymerase and either DTT or Penicillamine reducing agent. Reaction volumes across a type of reducing agent varied in their initial concentration of reducing agent. The second set of experiments was identical to the first, except that each reaction volume included a Deep Vent polymerase rather than a 9 degrees north polymerase. The reaction volumes in the first and second experimental sets were completed in bulk reaction volumes. The third experiments set included experiments each comprising a plurality of aqueous droplets generated with a 3 ng input of sample nucleic acid molecules, where each droplet included a polyacrylamide gel bead linked (via disulfide linkages) to oligonucleotides comprising barcode and priming sequences similar to oligonucleotides 308 shown in FIG. 3, sample nucleic acid molecules, a 9 degrees north polymerase and either DTT or Penicillamine reducing agent. Experiments across a given reducing agent varied in their initial concentration of the reducing agent.

Each set of experiments was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. For the third set of experiments conducted in droplets, the emulsion comprising the droplets was broken and the amplification products pooled into a common mixture. The recovered amplification products from each experimental set were then measured.

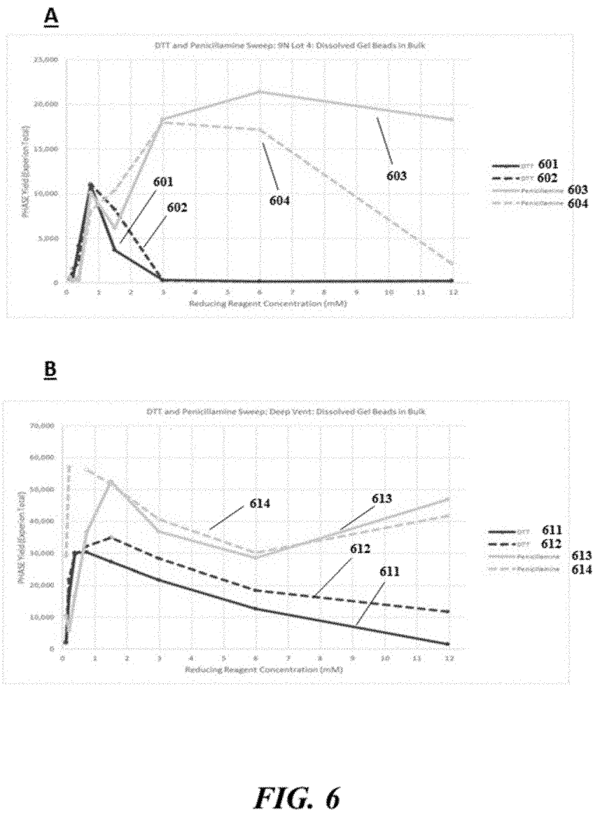

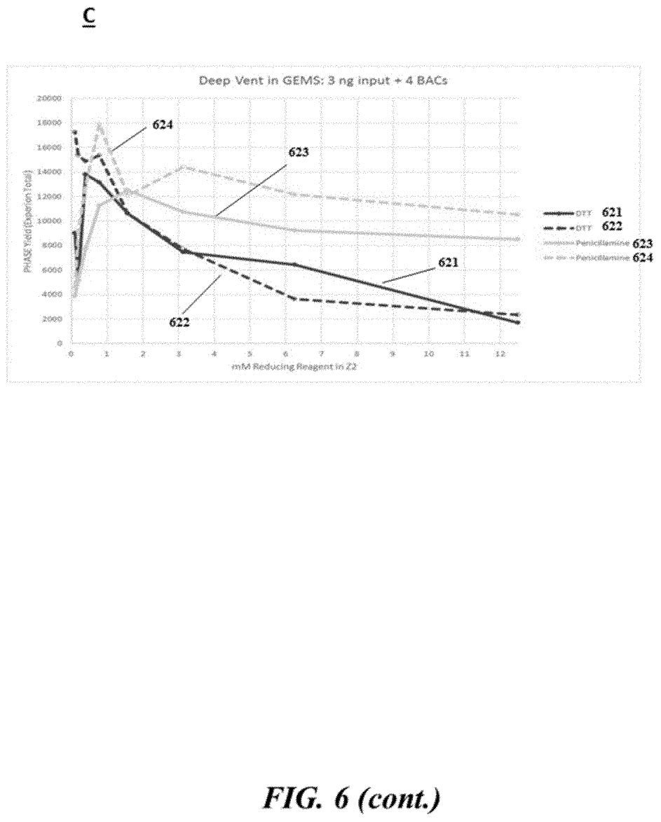

Amplification product yield versus initial reducing agent concentration for the various experimental sets are graphically depicted in FIG. 6. FIG. 6 (panel A) graphically depicts results from the first experimental set (601 and 602 corresponding to DTT replicates, 603 and 604 corresponding to Penicillamine replicates); FIG. 6 (panel B) graphically depicts results from the second experimental set (611 and 612 corresponding to DTT replicates, 613 and 614 corresponding to Penicillamine replicates); FIG. 6 (panel C) graphically depicts results from the third experimental set (621 and 622 corresponding to DTT replicates, 623 and 624 corresponding to Penicillamine replicates). For each experimental set, DTT replicates showed higher amounts of amplification products at generally lower initial concentrations of reducing agent, with substantially lower amounts of amplification products at higher initial concentrations.

Conversely, in all three experimental sets, amplification products generated using Penicillamine varied some with initial concentration of reducing agent, however produced amplification products in amounts comparable or better than amounts produced with DTT and, unlike DTT, in relatively similar amounts across the range of initial Penicillamine concentrations tested. Data indicate that Penicillamine can yield amplification products in similar amount across a broad range of initial reducing agent concentration when compared to other reducing reagents such as DTT. Such differences may be attributable the stabilized nature (e.g., via its sterically hindered thiol group) of Penicillamine, as described elsewhere herein.

### Example 4: Effect of Reducing Agent Type and Amplification Product Yield

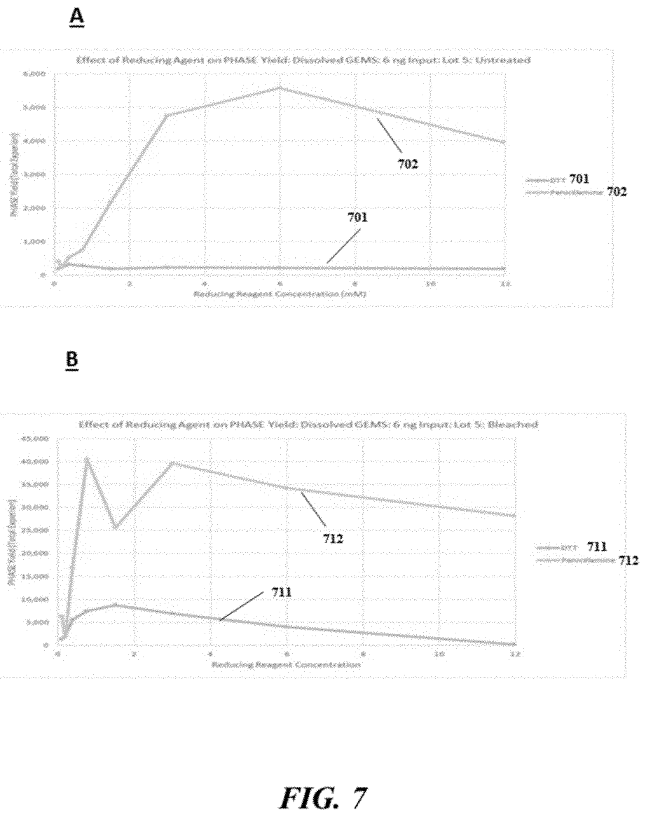

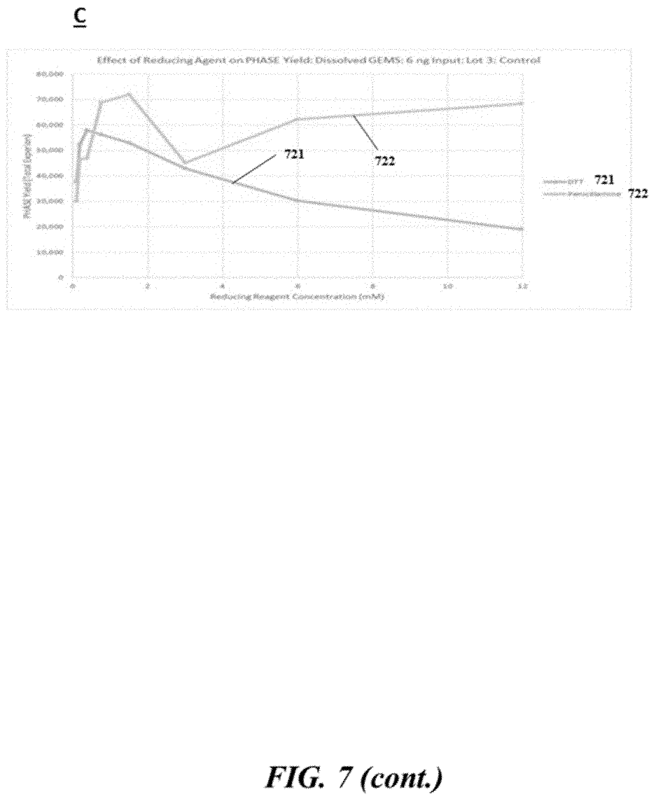

Three sets of experiments were performed to compare the performance of DTT and Penicillamine in generating amplification products in an amplification reaction. Each experiment included a plurality of aqueous droplets generated with a 6 ng input of sample nucleic acid molecules, where each droplet included a polyacrylamide gel bead linked (via disulfide linkages) to oligonucleotides comprising barcode and priming sequences similar to oligonucleotides 308 shown in FIG. 3, sample nucleic acid molecules, a 9 degrees north polymerase and either DTT or Penicillamine reducing agent. Each set of experiments included a different 9 degrees north polymerase preparation (a first preparation (“Lot 5: Untreated” in FIG. 7 panel A), a second preparation comprising oxidized bleached 9 degrees north derived from the first preparation (“Lot 5: Bleached” in FIG. 7 panel B) and a third preparation comprising a 9 degrees north preparation not derived from the first preparation (“Lot 3: Control” in FIG. 7 panel C). Moreover, droplets across experiments for a given reducing agent varied in their initial concentration of the reducing agent.

Each set of experiments was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. Following the completion of amplification reactions, the emulsion comprising the droplets was broken and the amplification products pooled into a common mixture for each experiment and the amount of recovered amplification products measured.

Amplification product yield versus initial reducing agent concentration for the various experimental sets are graphically depicted in FIG. 7. FIG. 7 (panel A) graphically depicts results from the first experimental set corresponding to the first 9 degrees north preparation (701 corresponding to DTT, 702 corresponding to Penicillamine); FIG. 7 (panel B) graphically depicts results from the second experimental set corresponding to the second 9 degrees north preparation (711 corresponding to DTT, 712 corresponding to Penicillamine); FIG. 7 (panel C) graphically depicts results from the third experimental set corresponding to the third 9 degrees north preparation (721 corresponding to DTT, 722 corresponding to Penicillamine). For each experimental set, DTT showed higher amounts of amplification products at generally lower initial concentrations of reducing agent, with lower amounts of amplification products at higher initial concentrations.

Conversely, in all three experimental sets, amplification products generated using Penicillamine varied some with initial concentration of reducing agent, however produced amplification products in amounts comparable or better than amounts produced with DTT and, unlike DTT, in relatively similar amounts across the range of initial Penicillamine concentrations tested. Data indicate that Penicillamine can yield amplification products in similar amount across a broad range of initial reducing agent concentration when compared to other reducing reagents such as DTT. Such differences may be attributable the stabilized nature (e.g., via its sterically hindered thiol group) of Penicillamine, as described elsewhere herein.

### Example 5: Effect of Reducing Agent Type and Amplification Product Yield

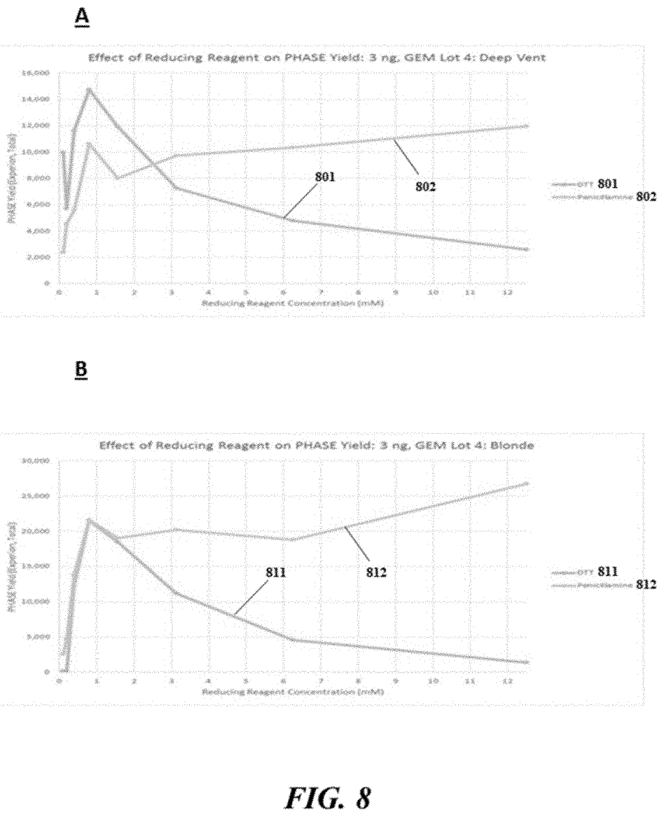

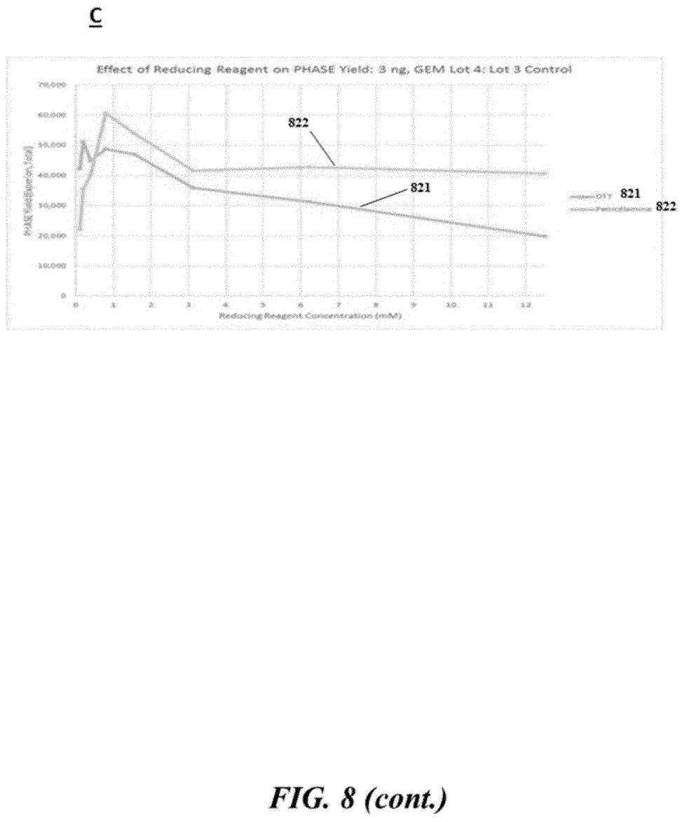

Three sets of experiments were performed to compare the performance of DTT and Penicillamine in generating amplification products in an amplification reaction. Each experiment included a plurality of aqueous droplets generated with a 3 ng input of sample nucleic acid molecules, where each droplet included a polyacrylamide gel bead linked (via disulfide linkages) to oligonucleotides comprising barcode and priming sequences similar to oligonucleotides 308 shown in FIG. 3, sample nucleic acid molecules, one of three polymerase preparations and either DTT or Penicillamine reducing agent. Each set of experiments included a different polymerase preparation (a Deep Vent polymerase preparation (“Deep Vent” in FIG. 8 panel A), an oxidized 9 degrees north preparation (“Blonde” in FIG. 8 panel B) and an untreated 9 degrees north preparation (“Control” in FIG. 8 panel C). Moreover, droplets across experiments for a given reducing agent varied in their initial concentration of the reducing agent.

Each set of experiments was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. Following the completion of amplification reactions, the emulsion comprising the droplets was broken and the amplification products pooled into a common mixture for each experiment and the amount of recovered amplification products measured.

Amplification product yield versus initial reducing agent concentration for the various experimental sets are graphically depicted in FIG. 8. FIG. 8 (panel A) graphically depicts results from the first experimental set corresponding to the Deep Vent preparation (801 corresponding to DTT, 802 corresponding to Penicillamine); FIG. 8 (panel B) graphically depicts results from the second experimental set corresponding to the oxidized 9 degrees north preparation (811 corresponding to DTT, 812 corresponding to Penicillamine); FIG. 8 (panel C) graphically depicts results from the third experimental set corresponding to the untreated 9 degrees north preparation (821 corresponding to DTT, 822 corresponding to Penicillamine). For each experimental set, DTT showed higher amounts of amplification products at generally lower initial concentrations of reducing agent, with lower amounts of amplification products at higher initial concentrations.

Conversely, in all three experimental sets, amplification products generated using Penicillamine varied some with initial concentration of reducing agent, however produced amplification products in amounts comparable or better than amounts produced with DTT and, unlike DTT, in relatively similar amounts across the range of initial Penicillamine concentrations tested. Data indicate that Penicillamine can yield amplification products in similar amount across a broad range of initial reducing agent concentration when compared to other reducing reagents such as DTT. Such differences may be attributable the stabilized nature (e.g., via its sterically hindered thiol group) of Penicillamine, as described elsewhere herein.

### Example 6: Effect of Reducing Agent Type and Amplification Product Yield

Three sets of experiments were performed to compare the performance of DTT and Penicillamine in generating amplification products in an amplification reaction. Each set of experiments included replicate experiments that included a plurality of aqueous droplets, where each droplet included a polyacrylamide gel bead linked (via disulfide linkages) to oligonucleotides comprising barcode and priming sequences similar to oligonucleotides 308 shown in FIG. 3, sample nucleic acid molecules, one of three polymerase preparations and either DTT or Penicillamine reducing agent. Each set of experiments included a different combination of polymerase preparation and reducing agent (the first set comprising an untreated 9 degrees north polymerase preparation and DTT, the second set comprising an oxidized 9 degrees north polymerase preparation and DTT and the third set comprising the oxidized 9 degrees north polymerase preparation and Penicillamine). Moreover, experiments across an experimental set varied in their initial concentration of the reducing agent.

Each set of experiments was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. Following the completion of amplification reactions, the emulsion comprising the droplets was broken and the amplification products pooled into a common mixture for each experiment and the amount of recovered amplification products measured.

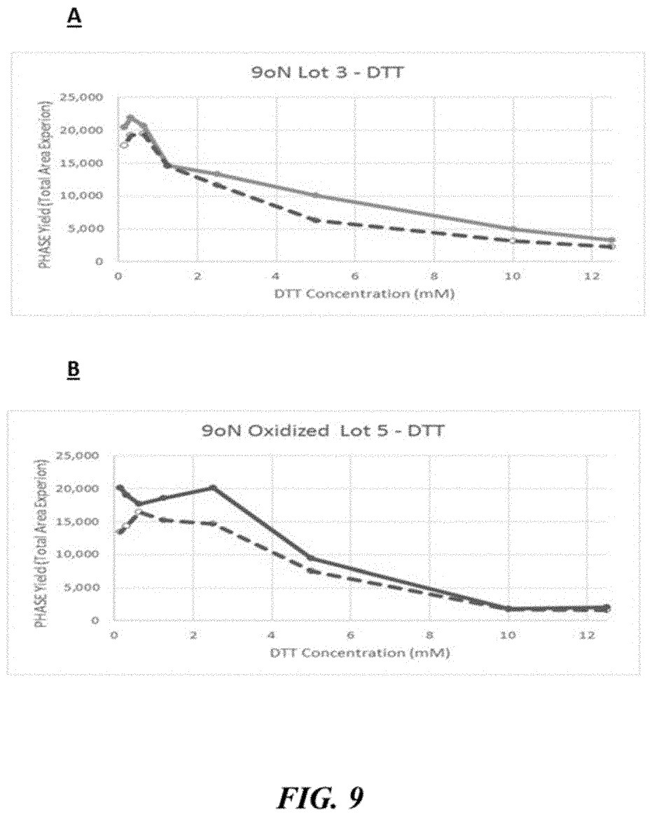

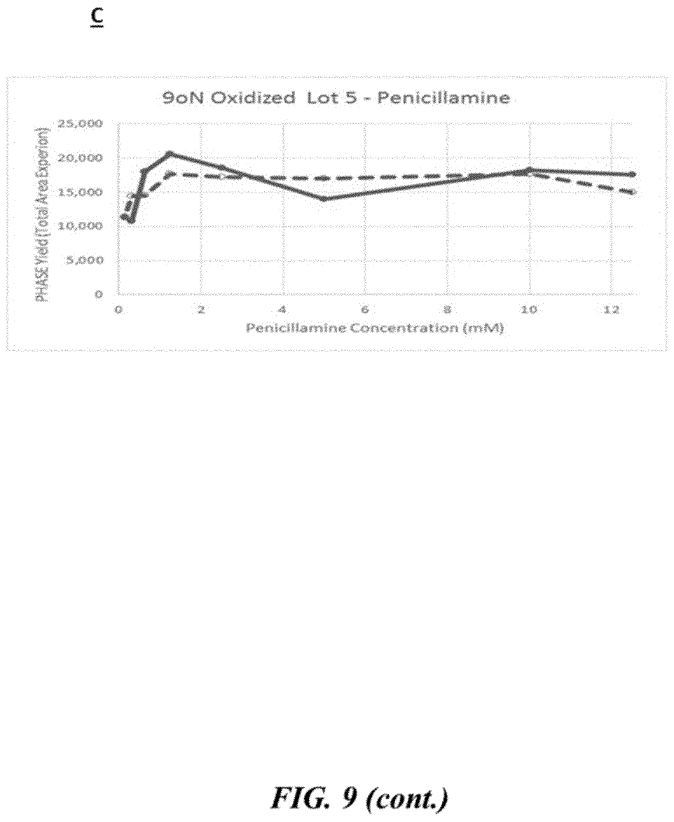

Amplification product yield versus initial reducing agent concentration for the various experimental sets are graphically depicted in FIG. 9. FIG. 9 (panel A) graphically depicts replicate results (solid and dashed lines) from the first experimental set corresponding to the untreated 9 degrees north preparation and DTT; FIG. 9 (panel B) graphically depicts replicate results (solid and dashed lines) from the second experimental set corresponding to the oxidized 9 degrees north preparation and DTT; FIG. 9 (panel C) graphically depicts replicate results (solid and dashed lines) from the first experimental set corresponding to the oxidized 9 degrees north preparation and Penicillamine. For the first and second experimental sets, DTT showed higher amounts of amplification products at generally lower initial concentrations of reducing agent, with lower amounts of amplification products at higher initial concentrations.

Conversely, in the third experimental set, amplification products generated using Penicillamine varied some with initial concentration of reducing agent, however produced amplification products in amounts comparable or better than amounts produced with DTT in the first and second sets and, unlike DTT, in relatively similar amounts across the range of initial Penicillamine concentrations tested. Data indicate that Penicillamine can yield amplification products in similar amount across a broad range of initial reducing agent concentration when compared to other reducing reagents such as DTT. Such differences may be attributable the stabilized nature (e.g., via its sterically hindered thiol group) of Penicillamine, as described elsewhere herein.

### Example 7: Effect of Reducing Agent Type and Amplification Product Yield

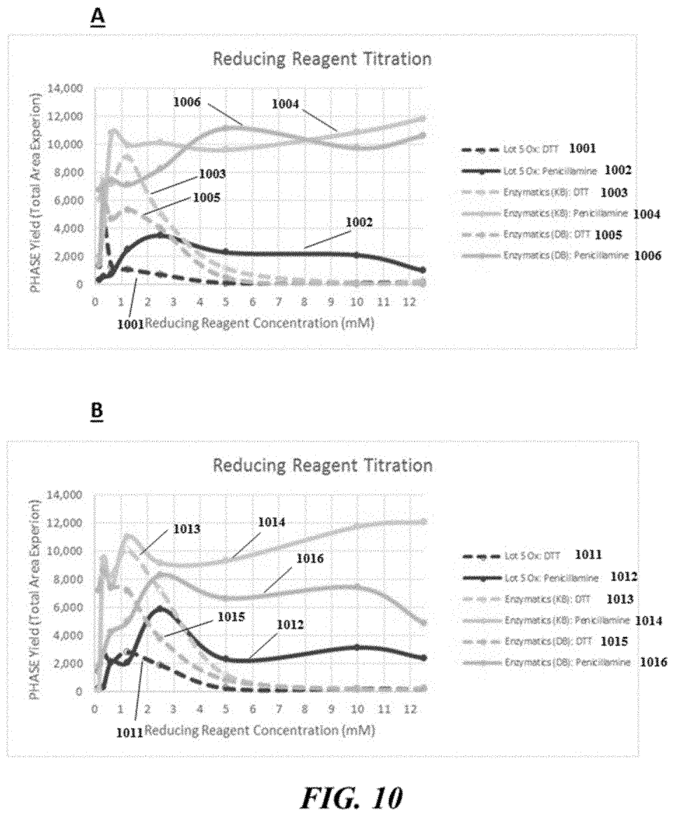

In duplicate, three sets of experiments were performed to compare the performance of DTT and Penicillamine in generating amplification products in an amplification reaction. Each experiment included a plurality of aqueous droplets, where each droplet included a polyacrylamide gel bead linked (via disulfide linkages) to oligonucleotides comprising barcode and priming sequences similar to oligonucleotides 308 shown in FIG. 3, sample nucleic acid molecules, one of three polymerase preparations and either DTT or Penicillamine reducing agent. Each set of experiments included a different polymerase preparation (a first oxidized 9 degrees north polymerase preparation (“Lot 5 Ox” in FIG. 10 panels A and B), a second untreated 9 degrees north preparation (“Enzymatics (KB)” in FIG. 10 panels A and B) and a third untreated 9 degrees north preparation (“Enzymatics (DB)” in FIG. 10 panels A and B). Moreover, droplets across experiments for a given reducing agent varied in their initial concentration of the reducing agent.

Each set of experiments was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. Following the completion of amplification reactions, the emulsion comprising the droplets was broken and the amplification products pooled into a common mixture for each experiment and the amount of recovered amplification products measured.

Amplification product yield versus initial reducing agent concentration for the various experimental sets are graphically depicted in FIG. 10. FIG. 10 (panel A) graphically depicts results for the first duplicate of experiments and FIG. 10 (panel B) graphically depicts results from the second duplicate of experiments. As shown in FIG. 10 (panel A) for each experimental set of the first duplicate, DTT (1001 for first oxidized 9 degrees north polymerase preparation, 1003 for second untreated 9 degrees north polymerase preparation, 1005 for third untreated 9 degrees north polymerase preparation) showed higher amounts of amplification products at generally lower initial concentrations of reducing agent, with lower amounts of amplification products at higher initial concentrations. As shown in FIG. 10 (panel B), similar results were obtained for the second duplicate of experiments when DTT was used as a reducing reagent (1011 for first oxidized 9 degrees north polymerase preparation, 1013 for second untreated 9 degrees north polymerase preparation, 1015 for third untreated 9 degrees north polymerase preparation).

Conversely, as shown in FIG. 10 (panel A) for all three experimental sets in the first duplicate of experiments, amplification products generated using Penicillamine (1002 for first oxidized 9 degrees north polymerase preparation, 1004 for second untreated 9 degrees north polymerase preparation, 1006 for third untreated 9 degrees north polymerase preparation) varied some with initial concentration of reducing agent, however produced amplification products in amounts comparable or better than amounts produced with DTT and, unlike DTT, in relatively similar amounts across the range of initial Penicillamine concentrations tested. Similar results were obtained for the second duplicate of experiments when Penicillamine was used as a reducing agent (1012 for first oxidized 9 degrees north polymerase preparation, 1014 for second untreated 9 degrees north polymerase preparation, 1016 for third untreated 9 degrees north polymerase preparation).

Data indicate that Penicillamine can yield amplification products in similar amount across a broad range of initial reducing agent concentration when compared to other reducing reagents such as DTT. Such differences may be attributable the stabilized nature (e.g., via its sterically hindered thiol group) of Penicillamine, as described elsewhere herein.

### Example 8: Effect of Reducing Agent Type on Rate of Chimera Observed During Nucleic Acid Sequencing

Two sets of experiments were performed to compare the propensities of DTT and Penicillamine, as part of a reaction scheme comprising an amplification reaction, to generate amplification products having observable chimera during nucleic acid sequencing of the amplification products. Each set of experiments included experiments having a plurality of aqueous droplets, where each droplet included a polyacrylamide gel bead linked (via disulfide linkages) to oligonucleotides comprising barcode and priming sequences similar to oligonucleotides 308 shown in FIG. 3, sample nucleic acid molecules, a polymerase preparation (e.g., 9 degrees north, oxidized 9 degrees north) and a reducing agent. The experimental sets differed in reducing agent, one set including DTT, the other set including Penicillamine. Across experiments in a given experimental set, various initial concentrations of reducing agent were tested.

Each experiment was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. Following the completion of amplification reactions, the emulsion comprising the droplets was broken and the amplification products pooled into a common mixture for each experiment. The pooled amplification products for each experiment were purified and subjected to a shear ligation process to add additional functional sequences to the amplification products and the further processed amplification products were then sequenced on a nucleic acid sequencer.

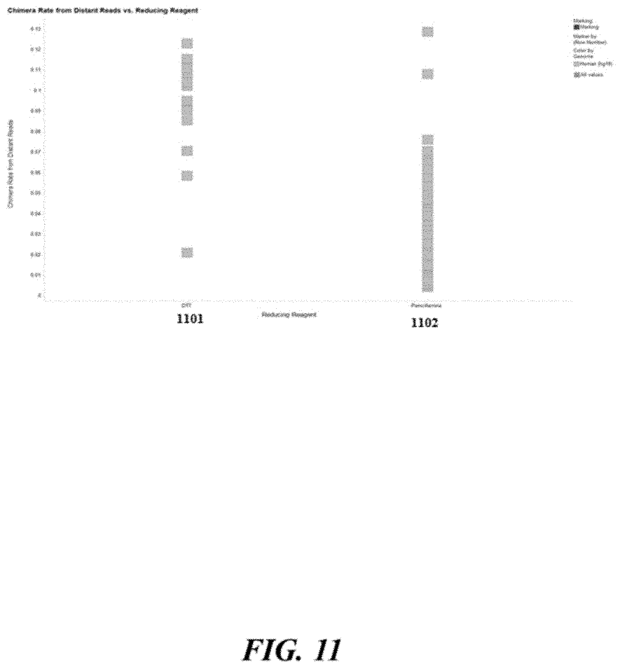

Observed chimera rate for the various experiments in each experimental set are graphically depicted in FIG. 11. As shown in FIG. 11, data obtained for various initial concentrations of DTT (1101 in FIG. 11) show a range of observed chimera rate, with relatively high chimera rates across the range of initial concentrations of DTT tested. Conversely, as is also shown in FIG. 11, data obtained for various initial concentrations Penicillamine (1102 in FIG. 11) show lower observed chimera rates when compared with data obtained from DTT and in a generally narrower range of chimera rates, over the range of initial concentrations of Penicillamine tested. Accordingly, data indicate that use of a stabilized reducing agent, such as Penicillamine, in a reaction scheme that generates amplification products can reduce the rate of chimera and/or variability in chimera rate that are observed in downstream sequencing of the amplification products.

### Example 9: Effect of Reducing Agent Type on Rate of Chimera Observed During Nucleic Acid Sequencing

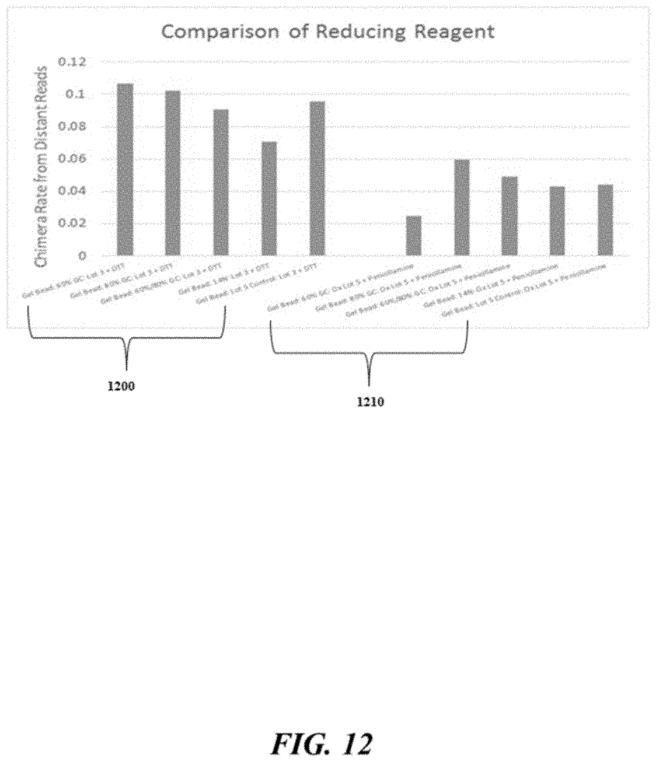

Two sets of experiments were performed to compare the propensities of DTT and Penicillamine, as part of a reaction scheme comprising an amplification reaction, to generate amplification products having observable chimera during nucleic acid sequencing of the amplification products. Each set of experiments included experiments having a plurality of aqueous droplets, where each droplet included a polyacrylamide gel bead linked (via disulfide linkages) to oligonucleotides comprising barcode and priming sequences similar to oligonucleotides 308 shown in FIG. 3, sample nucleic acid molecules, a polymerase preparation (e.g., 9 degrees north, oxidized 9 degrees north) and a reducing agent. The experimental sets differed in reducing agent, one set including DTT (1200 in FIG. 12), the other set including Penicillamine (1210 in FIG. 12). Each experimental set also utilized a different polymerase preparation (9 degrees north (“Lot 3” as shown in 1200 of FIG. 12) or oxidized 9 degrees north (“Ox Lot 5” as shown in 1210 of FIG. 12)). Across experiments within each experimental set, polyacrylamide beads varied in their source/preparation and/or the guanine-cytosine content (G-C content) of coupled oligonucleotides.

Each experiment was subjected to thermal cycling to amplify the sample nucleic acid molecules in an amplification reaction similar to the example amplification method graphically depicted in FIG. 3. During the amplification reactions, the appropriate reducing reagent reduced the disulfide linkages between oligonucleotides and beads, initiating the amplification reaction. Following the completion of amplification reactions, the emulsion comprising the droplets was broken and the amplification products pooled into a common mixture. The amplification products were purified and subject to a shear ligation process to add additional functional sequences to the amplification products and the further processed amplification products were then sequenced on a nucleic acid sequencer.

Observed chimera rate for the various experiments in each experimental set are graphically depicted in FIG. 12. As shown in FIG. 12, data obtained for of DTT experiments (1200 in FIG. 12) show higher chimera rates when compared to data obtained for Penicillamine experiments (1210 in FIG. 12).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-51. (canceled)

52. A method for processing a nucleic acid molecule, comprising:
(a) providing a reaction volume comprising said nucleic acid molecule, a reagent, and an agent that comprises a sterically hindered —SH group, wherein said reagent comprises a first component coupled to a second component via a linker, and wherein said linker comprises a cleavable moiety; and
(b) subjecting said reaction volume to conditions sufficient for said agent to cleave said cleavable moiety, thereby separating said first component from said second component.

53. The method of claim 52, wherein said —SH group of said agent is bound to a carbon atom comprising two or more alkyl groups.

54. The method of claim 52, wherein subjecting said reaction volume to said conditions comprises increasing a temperature of said reaction volume.

55. The method of claim 52, wherein said first component comprises a nucleic acid barcode molecule.

56. The method of claim 52, wherein said second component comprises a bead.

57. The method of claim 52, wherein said cleavable moiety is a disulfide linkage.

58. The method of claim 52, wherein said reaction volume is within an aqueous droplet.

59. A reaction volume comprising: a nucleic acid molecule, a reagent, and an agent that comprises a sterically hindered —SH group, wherein said reagent comprises a first component coupled to a second component via a linker, and wherein said linker comprises a cleavable moiety that is configured such that when said reaction volume is subjected to conditions sufficient for said agent to cleave said cleavable moiety, said first component separates from said second component.

60. The reaction volume of claim 59, wherein said —SH group of said agent is bound to a carbon atom comprising two or more alkyl groups.

61. The reaction volume of claim 59, wherein said agent is configured to cleave said cleavable moiety, thereby separating said first component from said second component.

62. The reaction volume of claim 59, wherein said first component comprises a nucleic acid barcode molecule.

63. The reaction volume of claim 59, wherein said second component comprises a bead.

64. The reaction volume of claim 59, wherein said cleavable moiety is a disulfide linkage.

65. The reaction volume of claim 59, wherein said reaction volume is within an aqueous droplet.

66. A method for processing a nucleic acid molecule, comprising:
(a) providing a reaction volume comprising said nucleic acid molecule, a reagent, and an agent in an inactive state, wherein said reagent comprises a first component coupled to a second component via a linker, wherein said agent comprises one or more —SH groups, and wherein said agent comprises a first cleavable moiety and said linker comprises a second cleavable moiety; and
(b) subjecting said reaction volume to conditions sufficient to cleave said first cleavable moiety of said agent to provide an activated agent that cleaves said second cleavable moiety of said linker, thereby separating said first component from said second component.

67. The method of claim 66, wherein subjecting said reaction volume to said conditions in (b) comprises increasing a temperature of said reaction volume.

68. The method of claim 66, wherein cleaving said first cleavable moiety of said agent comprises cleaving a covalent bond of said agent.

69. The method of claim 66, wherein said first cleavable moiety is an amide bond.

70. The method of claim 66, wherein said first component comprises a nucleic acid barcode molecule.

71. The method of claim 66, wherein said second component comprises a bead.

72. The method of claim 66, wherein said second cleavable moiety is a disulfide linkage.

73. The method of claim 66, wherein said reaction volume is within an aqueous droplet.

74. A reaction volume comprising: a nucleic acid molecule, a reagent, and an agent in an inactive state, wherein said reagent comprises a first component coupled to a second component via a linker, wherein said agent comprises one or more —SH groups, and wherein said agent comprises a first cleavable moiety and said linker comprises a second cleavable moiety that are configured such that when said reaction volume is subjected to conditions sufficient for cleavage of said first cleavable moiety of said agent, an activated agent is provided that is configured to cleave said second cleavable moiety of said linker such that said first component separates from said second component.

75. The reaction volume of claim 74, wherein said first cleavable moiety is configured to be cleavable upon increasing a temperature of said reaction volume.

76. The reaction volume of claim 74, wherein cleavage of said first cleavable moiety comprises cleavage of a covalent bond of said agent.

77. The reaction volume of claim 74, wherein said first cleavable moiety is an amide bond.

78. The reaction volume of claim 74, wherein said first component comprises a nucleic acid barcode molecule.

79. The reaction volume of claim 74, wherein said second component comprises a bead.

80. The reaction volume of claim 74, wherein said second cleavable moiety is a disulfide linkage.

81. The reaction volume of claim 74, wherein said reaction volume is within an aqueous droplet.
